(19) United States
(12) Patent Application Publication
(10) Pub. No.: US 2026/0108654 A1
(43) Pub. Date: Apr. 23, 2026

(54) ADHESIVE MEDICAL FILMS INCORPORATING CATIONIC STEROIDAL ANTIMICROBIALS

(71) Applicant: Paul B. SAVAGE, Mapleton, UT (US)

(72) Inventor: Paul B. SAVAGE, Mapleton, UT (US)

(73) Assignee: BRIGHAM YOUNG UNIVERSITY, Provo, UT (US)

(21) Appl. No.: 19/364,848

(22) Filed: Oct. 21, 2025

Related U.S. Application Data
(60) Provisional application No. 63/710,458, filed on Oct. 22, 2024.

(51) Int. Cl. A61L 15/44; A61F 13/0246; A61L 15/24; A61L 15/26; A61L 15/58

(52) U.S. Cl. CPC A61L 15/44; A61F 13/0253; A61L 15/24; A61L 15/26; A61L 15/58; A61L 2300/222; A61L 2300/406

(57) ABSTRACT

Disclosed are adhesive medical films incorporating one or more cationic steroidal antimicrobial (CSA) compounds. The adhesive medical film is in the form of a tape, dressing, or sheet, configured for contact against dermal tissue to provide securement of medical devices, wound care, wound closure, and/or scar prevention. The adhesive medical film includes (i) a polymer adhesive (e.g., pressure-sensitive adhesive) in which one or more CSA compounds are distributed, and (ii) optionally, a backing substrate upon which the polymer adhesive is disposed. Examples of polymer adhesives include polyacrylate and silicone adhesives. The adhesive medical films, when used, reduce microbial colonization and/or growth, both on the films during storage and at a dermal site, as compared to the same adhesive medical film without incorporated CSA compounds.

### CROSS-REFERENCE TO RELATED APPLICATIONS

This application claims the benefit of U.S. Provisional Application No. 63/710,458, filed Oct. 22, 2024, which is incorporated by reference in its entirety.

### BACKGROUND

Healthcare-associated infections (HAIs) involving surgical sites, skin trauma, and devices penetrating the skin are a frequent source of increased expense, hospitalization periods, and adverse outcomes. As of 2022, HAIs such as central line-associated bloodstream infections and skin and soft tissue infections are responsible for 72,000 patient deaths annually in the United States. Surgical site infections are common and frequently lead to hospital readmission with associated mortality in as many as 5% of cases. Many common HAI pathogens, including Candida albicans (CA), Pseudomonas aeruginosa (PA), and methicillin-resistant Staphylococcus aureus (MRSA), are considered as urgent or serious threats. MRSA alone is globally responsible for 100,000 deaths annually as well as almost $2 billion dollars in health care costs.

Medical adhesives are often employed to help protect compromised skin from infection and/or to secure medical devices, but adhesives can become contaminated by pathogens, exposing wounds, surgical sites, and medical devices to colonization. Medical adhesives are ubiquitous in healthcare facilities and are used to secure devices and lines extending through the skin and to protect compromised skin from microbial exposure and/or to reduce surgical scarring. However, these devices are also vectors for cross-contamination, and microbes such as PA, MRSA, and CA too often contaminate adhesive devices like cardiac tape and cord catheters.

Because of this risk, bacteria such as PA and Staphylococcus aureus (SA) have frequently been cultured from medical adhesives. Fungal pathogens such as Aspergillus flavus (AF) (and CA have also been cultured from adhesive devices such as cardiac tape and cord catheters, with counts on covered skin being over one thousand times higher than control sites in some instances. The problem is widespread, and some studies have found that as much as 74% of catheter tape and 68% of operating room tape are colonized with microbes before they are used in treating patients. This confirms that these tapes can contaminate wound and surgical sites, leading to unnecessary infections at healthcare facilities.

Accordingly, there is an ongoing need for medical adhesive materials that can reduce the incidence and/or severity of infections such as HAIs.

### SUMMARY

Disclosed herein are adhesive medical films incorporating one or more cationic steroidal antimicrobial (CSA) compounds. The adhesive medical films can be in the form of a tape, dressing, or sheet, for example, configured for contact against dermal tissue to provide securement of medical devices, wound care, wound closure, and/or scar prevention. The adhesive medical films can include (i) a polymer adhesive (e.g., pressure-sensitive adhesive) in which one or more CSA compounds are distributed, and (ii) optionally, a backing substrate upon which the polymer adhesive is disposed, the backing substrate preferably being free of CSA compounds. The adhesive medical films, when used, reduce microbial colonization and/or growth as compared to the same adhesive medical film without the incorporated CSA compounds. Moreover, they are not prone to development of microbial resistance, which could still permit microbial growth.

Also disclosed are methods of manufacturing adhesive medical films incorporating one or more CSA compounds. In one embodiment, a polyacrylate-based adhesive medical film can be manufactured by a method comprising: (i) mixing one or more CSA compounds with an organic solvent (e.g., an organic alcohol such as ethanol) to form a CSA liquid; (ii) mixing the CSA liquid with a polymer adhesive composition, wherein the polymer adhesive composition comprises polyacrylate, to form a CSA adhesive mixture; applying the CSA adhesive mixture onto a releasable substrate; and (iii) allowing the CSA adhesive mixture to dry or at least partially solidify to form a polymer adhesive film incorporating the one or more CSA compounds and polyacrylate. The method can further comprise adding a backing substrate to an upper surface of the polymer adhesive after allowing the CSA adhesive mixture to dry or at least partially solidify. In another embodiment, a silicone-based adhesive medical film is manufactured by a method comprising: (i) mixing one or more CSA compounds with an organic alcohol or other appropriate solvent to form a CSA liquid; (ii) soaking a silicone film material in the CSA liquid such that the silicone film material swells and takes up at least a portion of the one or more CSA compounds of the CSA liquid; and (iii) optionally, sonicating the CSA liquid while the silicone film material is in the CSA liquid.

Also disclosed herein is a method of using an adhesive medical film comprising: (i) applying the adhesive medical film such that at least a portion contacts dermal tissue of a subject; and (ii) the adhesive medical film reducing microbial colonization and/or growth as compared to the same adhesive medical film without incorporated CSA compounds. The adhesive medical film preferably includes a pressure-sensitive adhesive that adheres by applying light or gentle pressure. Pressure-sensitive adhesives typically do not form a chemical bond with dermal tissue, facilitating their removal without damaging tissue when their use is finished.

This summary is provided to introduce a selection of concepts in a simplified form that are further described below in the detailed description. This summary is not intended to identify key features or essential features of the claimed subject matter, nor is it intended to be used as an indication of the scope of the claimed subject matter.

### BRIEF DESCRIPTION OF THE DRAWINGS

Various objects, features, characteristics, and advantages of the disclosure will become apparent and more readily appreciated from the following description, taken in conjunction with the accompanying drawings and the appended claims, all of which form a part of this specification. In the Drawings, like reference numerals may be utilized to designate corresponding or similar parts in the various Figures, and the various elements depicted are not necessarily drawn to scale, wherein:

FIG. 1A illustrates examples of CSA compounds having ester or amide linkages at one or more of the R₃, R₇, and R₁₂ positions;

FIG. 1B illustrates examples of CSA compounds having ether linkages at one or more of the R₃, R₇, and R₁₂ positions;

FIG. 1C illustrate example CSA compounds having an amide linkage within the R₁₈ group and ether or urethane linkages at the R₃, R₇, and R₁₂ positions;

FIG. 1D illustrate example CSA compounds having a glyceride linkage within the R₁₈ group and ester linkages at the R₃, R₇, and R₁₂ positions;

FIG. 2A shows total extraction of CSA compounds from tested pressure-sensitive adhesive medical films;

FIG. 2B shows 3-day rolling average of daily elution of CSA compounds from tested pressure-sensitive adhesive medical films;

FIGS. 2C and 2D show SEM imaging of tested medical adhesive films based on a pressure-sensitive polyacrylate adhesive, with release paper being visible above the adhesive and the substrate pad being visible below the adhesive;

FIGS. 3A-3I show daily growth of MRSA, PA, and CA in the presence of tested pressure-sensitive adhesive medical films infused with CSA-44 or CSA-131; polyacrylate-based films incorporating CSA-131 are labelled as POLYA131; polyacrylate-based films incorporating CSA-44 are labelled POLYA44; and silicone-based films incorporating CSA-131 are labelled as SCART131; unaltered adhesive film on the same substrate (POLYAC) or un-infused scar tape (SCARTC) were used as controls; tests were run in triplicate with error bars indicating standard deviation; *p<0.05 by Student's T test; limit of detection (10² CFU/mL) is indicated by horizontal line;

FIGS. 4A-4F show quantification of MRSA, PA, and CA on pressure-sensitive adhesives and on agar beneath adhesives applied to agar lawns of microorganisms; tests were run in triplicate with error bars indicating standard deviation; *p<0.05 by Student's T test; limit of detection (10² CFU/cm²) is indicated by a horizontal line; and

FIGS. 5A and 5B show impact of CSA compounds on shear adhesive strength, with FIG. 5A showing a schematic of the lap shear test and FIG. 5B showing shear strength measurements of POLYAC controls compared to POLYA44 or POLYA131; error bars represent standard deviation. *p<0.05.

### DETAILED DESCRIPTION

### I. Introduction

In addition to its natural flora, human skin is routinely exposed to diverse microbes. When a medical device is placed near or in contact with the skin, it becomes a nidus for microbial growth because medical adhesive materials, including polyacrylate- and silicone-based materials, can serve as abiotic surfaces that pathogens can colonize.

The disclosed medical adhesive materials incorporating CSA compounds can beneficially reduce medical adhesive contamination and associated infections. The medical adhesive materials can controllably elute CSA compounds for over two weeks under conditions of use, providing antimicrobial activity against gram-positive bacteria such as methicillin-resistant Staphylococcus aureus (MRSA), gram-negative bacteria such as Pseudomonas aeruginosa (PA), and pathogenic fungi such as Candida albicans (CA). Moreover, incorporating CSA compounds into an adhesive composition can maintain or even improve lap-shear adhesive strength compared to the same adhesive composition without the CSA compounds. Further, the adhesive comprising CSA compounds can avoid dermal irritation when used in vivo.

An adhesive medical film as disclosed herein can take the form of a tape, sheet, dressing, or the like. Such medical adhesive materials have a variety of applications in the medical setting, including medical device securement (e.g., for securing dressings, bandages, intravenous lines, electrodes, catheters, ostomy bags, insulin pumps, prosthetic/orthotic devices), wound care and/or closure (e.g., incision closure, burn dressings), and scar prevention. A certain class of medical adhesive materials primarily used for scar prevention is often referred to as “scar tape”. Scar tape is placed over scars to flatten, soften, and reduce the appearance of the scar over time.

The medical adhesive materials and adhesive medical films preferably include a pressure-sensitive adhesive that adheres by applying light or gentle pressure. Pressure-sensitive adhesives typically do not form a chemical bond with dermal tissue, facilitating their removal with damaging tissue when their use is finished.

### II. Overview of CSA Compounds

CSA compounds, also referred to as “CSAs”, “CSA molecules”, or “ceragenin” compounds, are synthetically produced, small molecule chemical compounds that include a sterol backbone having various charged groups (e.g., amine, guanidine, and/or other cationic groups capable of exhibiting cationic properties under biological conditions) attached to the backbone. The sterol backbone can be used to orient the cationic groups on a face or plane of the sterol backbone. CSAs are cationic and amphiphilic, based upon the functional groups attached to the backbone. They are facially amphiphilic with a hydrophobic face and a polycationic face.

Example CSA compounds can have a structure of Formula I with appropriate substituents (R groups).

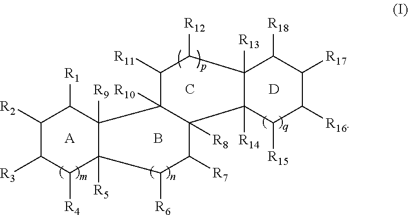

The CSA compounds can more particularly have a structure of Formula II, Formula III, or Formula IV:

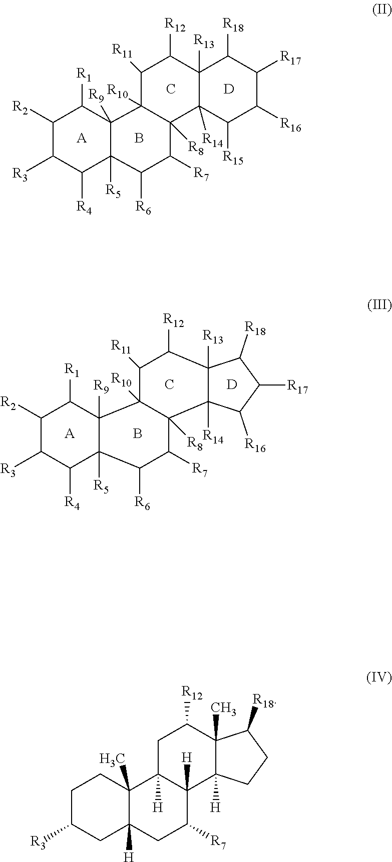

As will be discussed in greater detail below, the R groups of Formulae I, II, III and IV can have a variety of different functionalities, thus providing a given CSA compound with specific properties. The sterol backbone can be formed of 5-member and/or 6-member rings, so that p, q, m, and n in Formula I may independently be 1 (providing a 6-member ring) or 0 (providing a 5-member ring). Definitions for the R groups are set forth below.

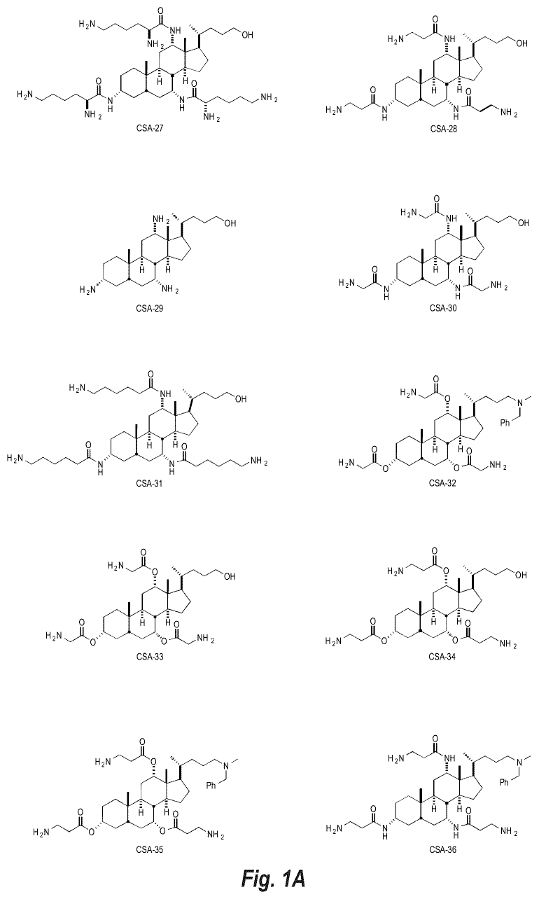

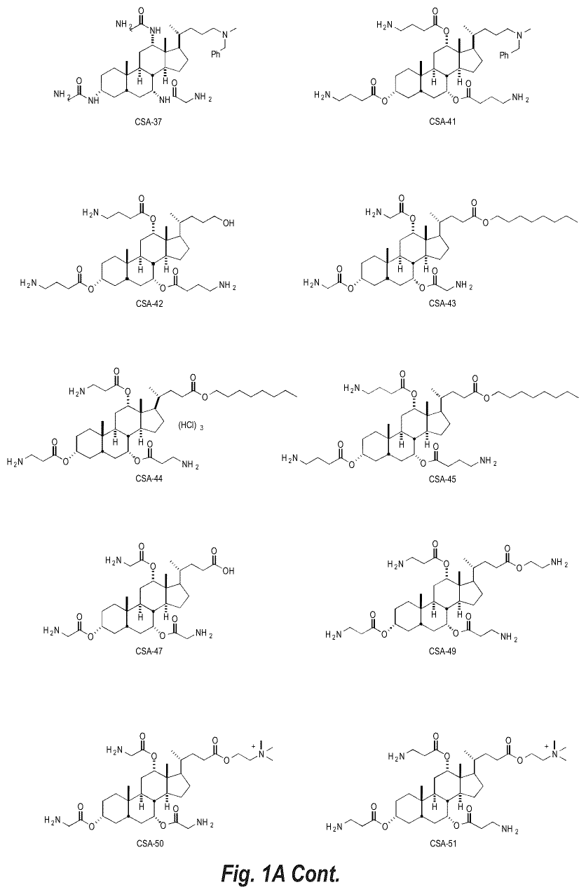

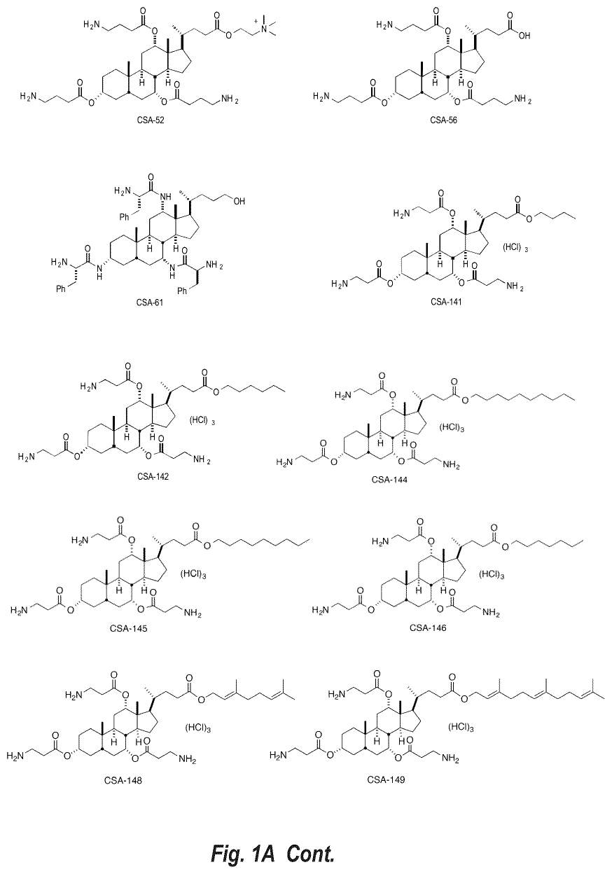

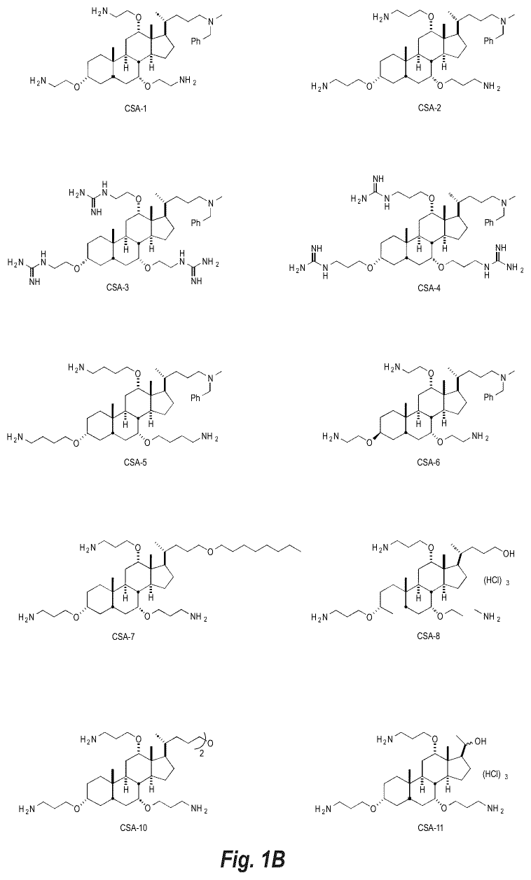

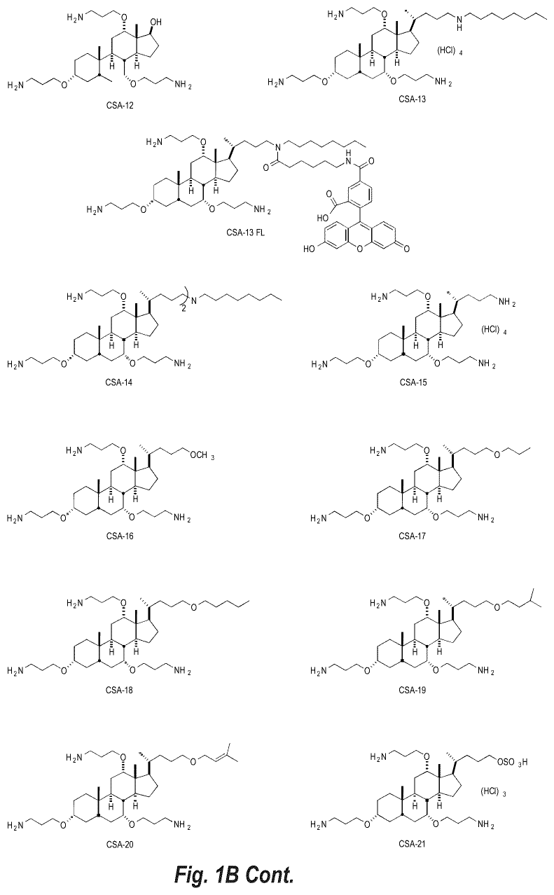

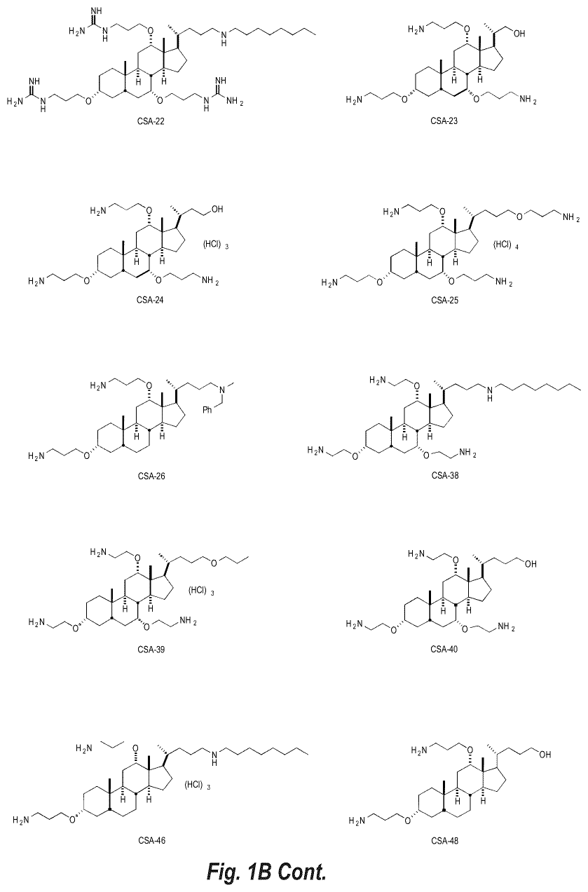

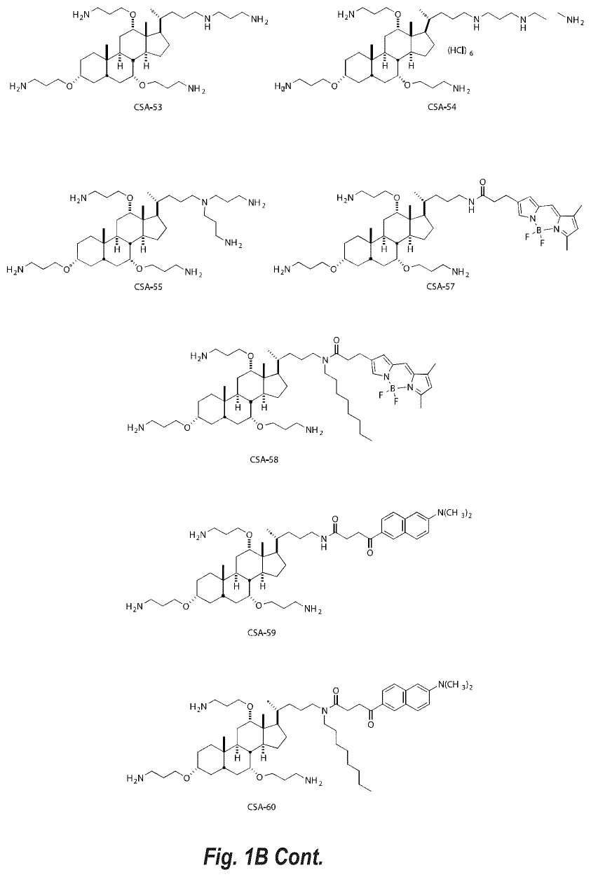

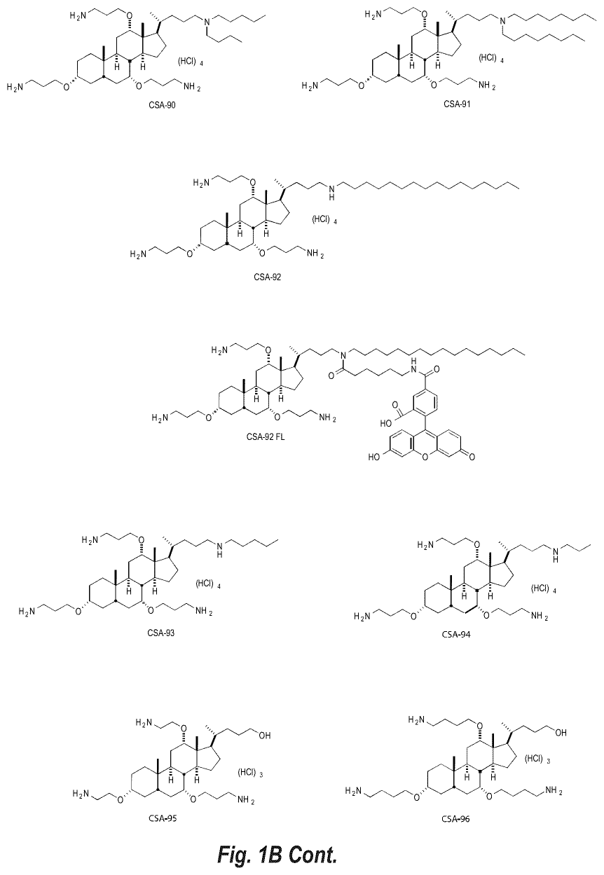

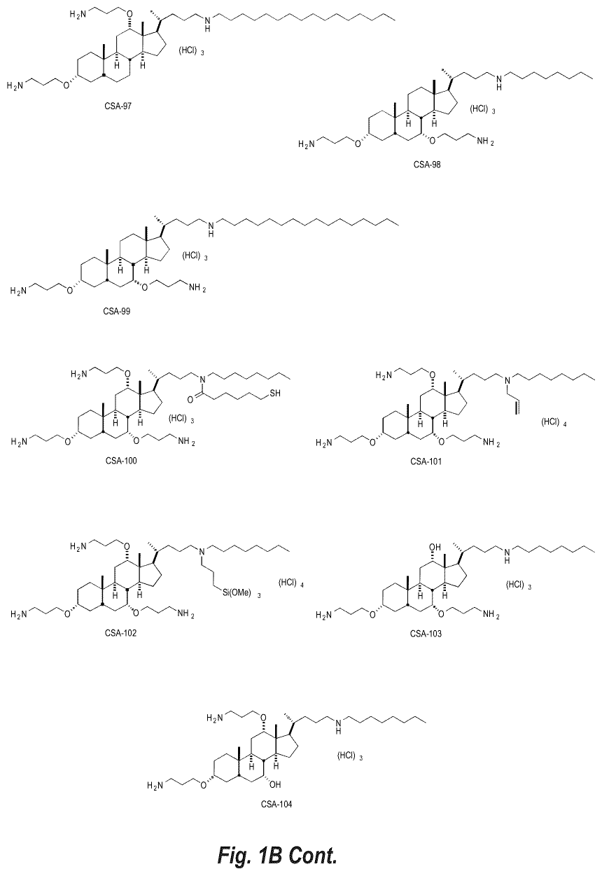

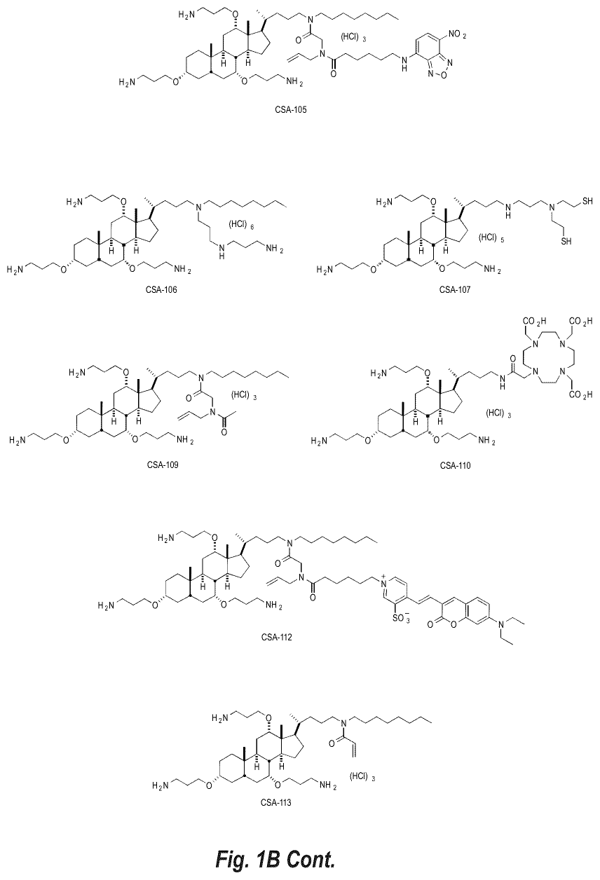

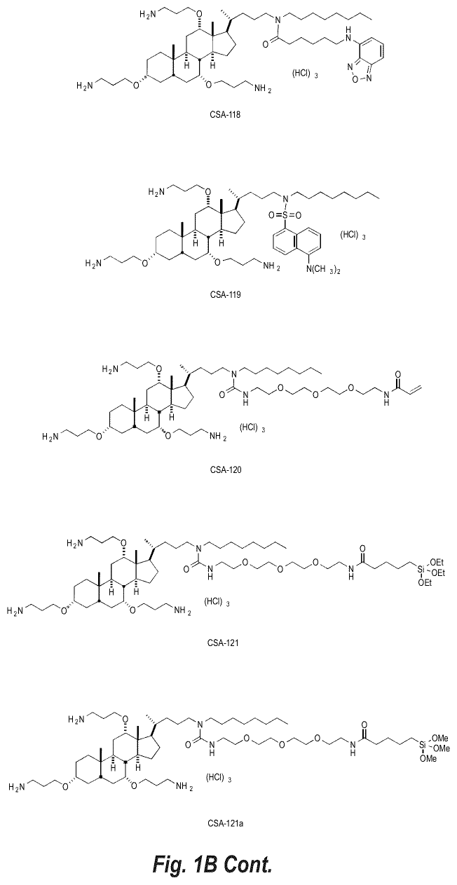

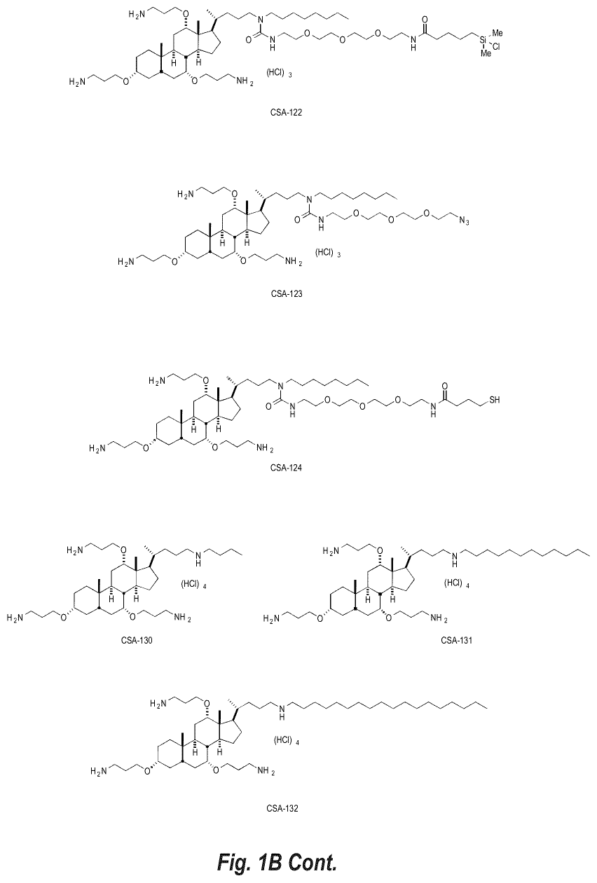

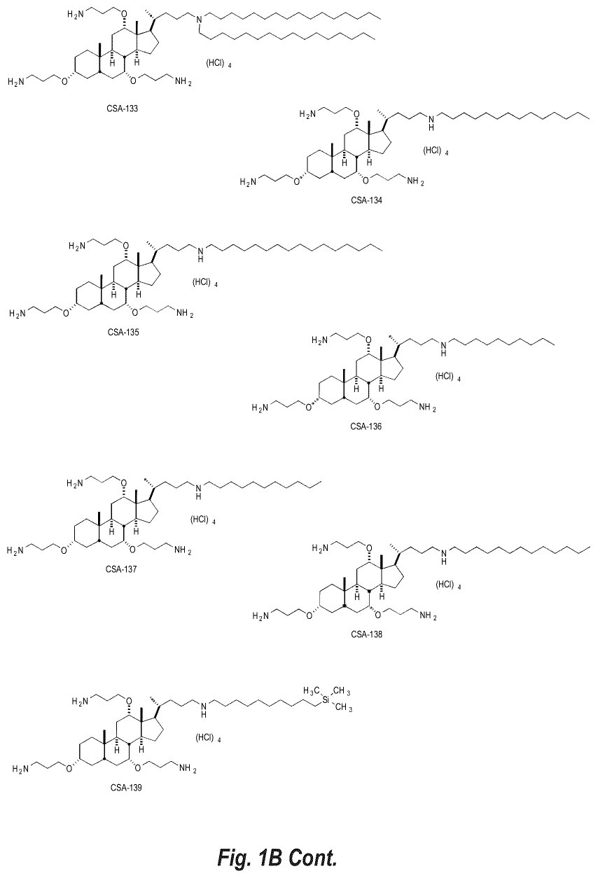

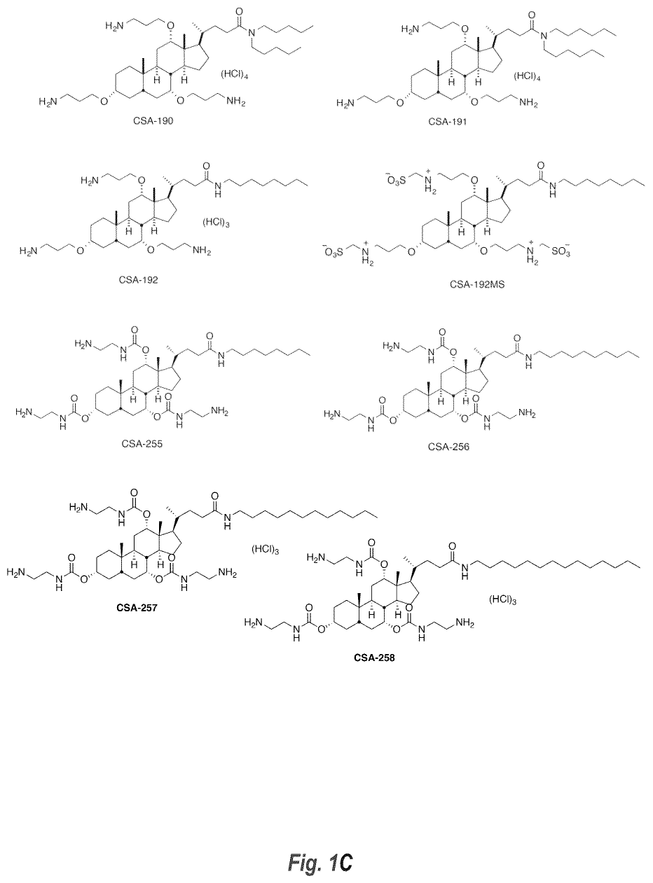

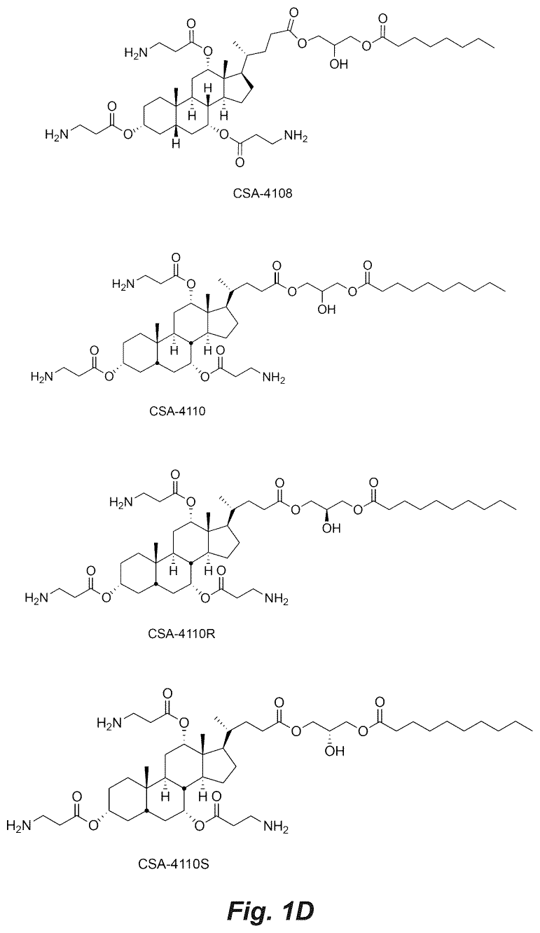

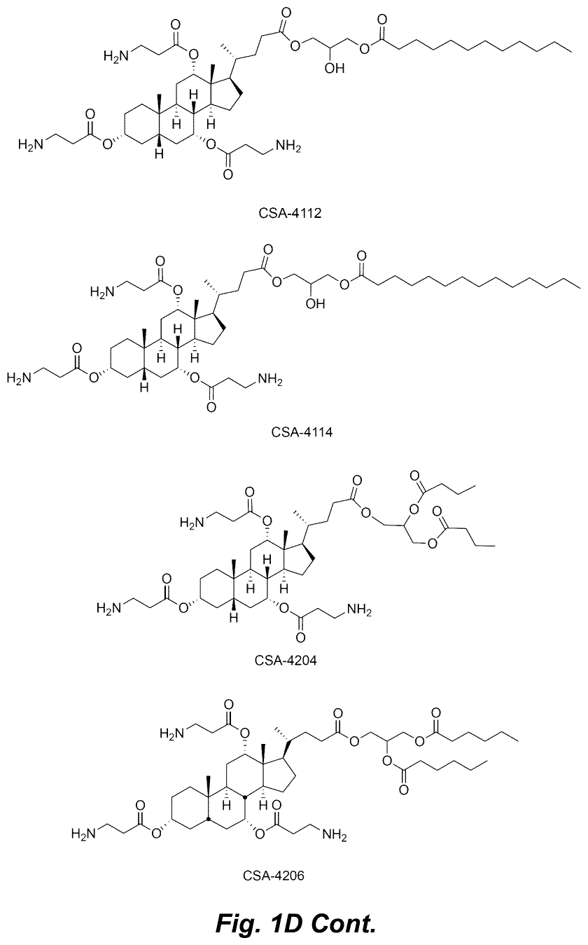

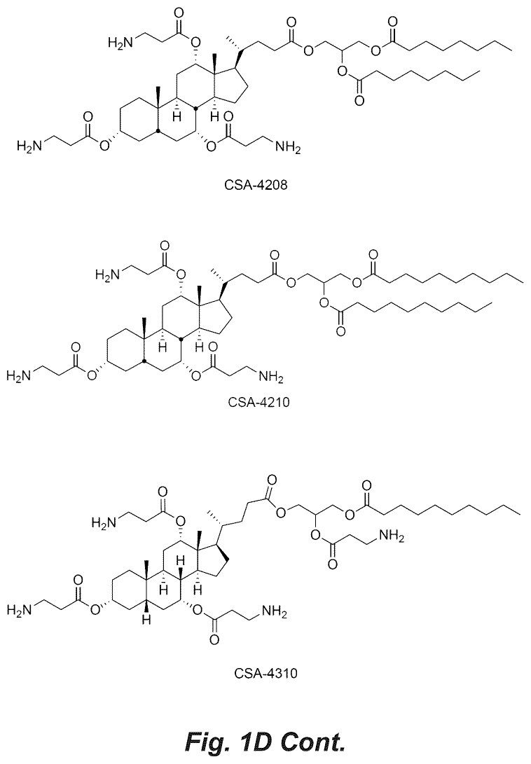

Formula II is a subset of Formula I in which rings A, B, C, and D are 6-member rings. Formula III is a subset of Formula I in which rings A, B, and C are 6-member rings and D is a 5-member ring. Formula IV is a subset of Formula III in which the stereochemistry is defined and the R groups other than R₃, R₇, R₁₂, and R₁₈ are defined as either hydrogen or methyl. Typically, the A, B, and C rings are 6-member rings and the D ring is a 5-member ring (e.g., Formulae III and IV). Examples of CSA compounds of Formula I, Formula II, Formula III, and Formula IV are illustrated in FIGS. 1A-1D.

Typically, CSAs used herein are of two types: (1) CSAs having cationic groups linked to the sterol backbone with hydrolysable linkages and (2) CSAs having cationic groups linked to the sterol backbone with non-hydrolysable linkages. For example, one type of hydrolysable linkage is an ester linkage, and one type of non-hydrolysable linkage is an ether linkage. CSAs of the first type can be “inactivated” by hydrolysis of the linkages coupling the cationic groups to the sterol backbone, whereas CSAs of the second type are more resistant to degradation and inactivation.

In preferred embodiments, CSA compounds of Formula I, Formula II, Formula III, and Formula IV are characterized by at least two of R₃, R₇, or R₁₂ independently including a cationic moiety attached to the sterol backbone via hydrolysable (e.g., ester) or non-hydrolysable (e.g., ether) linkages. A tail moiety is usually attached to Formula I at R₁₈. The tail moiety may be charged, uncharged, polar, non-polar, hydrophobic, or amphipathic, for example, and can thereby be selected to adjust the properties of the CSA and/or to provide desired characteristics. In a preferred embodiment of a CSA compound, at least two of R₃, R₇, or R₁₂ include a cationic moiety attached to the sterol backbone via non-hydrolysable (e.g., ether) linkages, such CSA-131.

The activity of the CSA compounds can be affected by the orientation of the substituent groups attached to the backbone structure. In one embodiment, the substituent groups attached to the backbone structure are oriented on a single face of the CSA compound. Accordingly, each of R₃, R₇, and R₁₂ may be positioned on a single face of Formula I, Formula II, Formula III, and Formula IV. In addition, R₁₈ may also be positioned on the same single face.

### III. Medical Adhesive Materials Incorporating CSA Compounds

An adhesive medical film as disclosed herein can take the form of a tape, sheet, dressing, or the like. The adhesive medical film includes (i) a polymer adhesive and (ii) optionally, a backing substrate upon which the polymer adhesive is disposed.

The polymer adhesive can be formulated as a pressure-sensitive adhesive (PSA). PSAs are adhesives that bond to surfaces when light pressure is applied, without requiring heat, solvents, or water. Pressure-sensitive adhesives facilitate application to dermal tissue using light or gentle pressure and removal without significantly damaging dermal tissue.

The polymer adhesive can include a polyacrylate. That is, the polymer adhesive can include one or more polyacrylate polymers formed (e.g., via free-radical polymerization) from a set of one or more acrylate or methacrylate (i.e., “(meth)acrylate”) monomers. The set of monomers can include, for example, an alkyl (meth)acrylate and/or a cycloalkyl (meth)acrylate. Additionally, or alternatively, the set of monomers can include a di(meth)acrylate, such as a di(meth)acrylate formed from a diol, such as 1,4 butanediol di(meth)acrylate. Additionally, or alternatively, the set of monomers can comprise a monomer with an acid group, such as a (meth)acrylic acid.

According to convention known to the skilled person, the terms “(meth)acrylic” and “(meth)acrylate” refer to both the acrylic/acrylate and methacrylic/methacrylate forms of the indicated compound. For example, (meth)acrylate monomers can include acrylate monomers (without the methyl substitution) and/or methacrylate monomers (with the methyl substitution). In addition, a “(meth)acrylic” or “(meth)acrylate” compound is inclusive of acrylic acid forms, acrylic acid anhydride forms, and derivatives thereof. Such derivatives include, for example, alkyl esters of acrylic acids, lower alkyl-substituted acrylic acids (e.g., C1-C2 substituted acrylic acids, such as methacrylic acid and/or ethyl substituted acrylic acid), and alkyl esters of lower alkyl-substituted acrylic acids (e.g., methyl methacrylate).

As used herein, an “alkyl (meth)acrylate” refers to an alkyl ester of a (meth)acrylic acid. An “alkyl” is a linear or branched (e.g., due to substitution) carbon chain functional group of the general formula CₙH₂ₙ₊₁ when positioned as a terminal group or CₙH₂ₙ when positioned as a non-terminal group (i.e., when the alkyl is within a larger molecule, and both ends of the alkyl are bonded to other carbons).

As used herein, a “cycloalkyl (meth)acrylate” refers to a cycloalkyl ester of a (meth)acrylic acid. A “cycloalkyl” is a carbon ring functional group (optionally with one or more substituents) of the general formula CₙH₂ₙ₋₁.

As used herein, a di(meth)acrylate refers to a compound that includes two (meth)acrylate groups. Such compounds can be formed by reacting (meth)acrylic acid with a diol, for example.

As used herein, a “substituted” compound is a compound in which at least one hydrogen has been replaced with a “substituent” group that is not hydrogen.

The backing substrate can include a flexible material suitable for medical use (e.g., biocompatible, non-irritating to dermal tissues, comfortable, breathable, hypoallergenic). Example backing substrate materials include woven fabrics (e.g., cotton, polyester, elastane, other textiles, blends thereof), nonwoven fabrics (e.g., spunlace fabrics, polyester, polypropylene, rayon, other textile fabrics, blends thereof), films (e.g., hydrocolloid films, polyurethane, polyethylene, polypropylene, silicone, other polymers, combinations thereof), polymer foam (e.g., polyurethane, polyethylene, other polymers, combinations thereof),

It has been found that the incorporation of one or more CSA compounds in the polymer adhesive does not measurably lower the lap shear strength of the adhesive. Surprisingly and unexpectedly, in certain embodiments, the incorporation of one or more CSA compounds in the polymer adhesive was determined to actually increase the lap shear strength of the polymer adhesive. When the one or more CSA compounds comprise a CSA compound that includes cationic groups linked to a sterol backbone with non-hydrolysable linkages (e.g., CSA-131), the lap shear strength of the polymer adhesive can be increased as compared to the same polymer adhesive without the incorporated CSA compounds. For example, the lap shear strength of the polymer adhesive can be increased by at least 1.5 times and up to 4.5 times as compared to the same polymer adhesive without incorporated CSA compounds. This is surprising, unexpected, and unpredictable. When the one or more CSA compounds comprise a CSA compound that includes cationic groups linked to a sterol backbone with hydrolysable linkages (e.g., CSA-44), the lap shear strength of the polymer adhesive was increased by about 12%.

In some embodiments, the polymer adhesive comprises a polysiloxane (i.e., silicone). The side groups of the polysiloxane can include H, alkyl (e.g., C1 to C6), phenyl, vinyl, amino, alkoxy (e.g., C1 to C6), and combinations thereof. An example includes polydimethylsiloxane (PDMS).

In some embodiments, the polymer adhesive includes a first side and second side, where the first side comprises capped polysiloxane and the second side comprises uncapped polysiloxane. Uncapped polysiloxanes are polysiloxane polymers that have reactive end groups, such as silanol or silyl acetate groups, which are not “capped” or terminated by other chemical groups. A silicone film with a first side of capped silicone and a second side of uncapped silicone can be configured so that the uncapped silicone side is the adhesive (tissue-facing) side.

The polymer adhesive can have an average thickness of about 50 μm to about 500 μm, or such as about 75 μm to about 300 μm, or such as about 100 μm to about 200 μm, or such as about 120 μm to about 150 μm, or a thickness within a range with endpoints selected from any two of the foregoing values.

The polymer adhesive can incorporate the one or more CSA compounds at a level of about 400 μg/cm² to about 3,000 μg/cm², or such as about 500 μg/cm² to about 2,000 μg/cm², or such as about 750 μg/cm² to about 1,500 μg/cm², or such as about 1,400 μg/cm², or a loading level within a range with endpoints selected from any two of the foregoing values.

In terms of volume, for a polymer adhesive thickness of 120 μm, for example, the CSA loading (or concentration) can be about 4.8 μg/cm³ to about 36 μg/cm³, or about 6 μg/cm³ to about 24 μg/cm³, or about 9 μg/cm³ to about 18 μg/cm³, or about 16.8 μg/cm³, or can be within a range with endpoints selected from any two of the foregoing values. Optionally, with different polymer adhesive thicknesses, the overall loading or concentration can be adjusted to achieve the foregoing volumetric concentrations.

As demonstrated by the Examples disclosed herein, polymer adhesives with such thicknesses and/or such CSA loading rates were able to controllably elute CSA compounds over sustained periods to effectively reduce microbial growth for multiple days (e.g., at least about 6 days, about 12 days, about 20 days, about 30 days, about 40 days, about 50 days, about 60 days, or up to about 68 days) relative to controls.

### IV. Methods of Manufacture

A polyacrylate-based adhesive medical film as disclosed herein can be manufactured by the steps of: (i) mixing one or more CSA compounds with an organic solvent (e.g., an organic alcohol such as ethanol) to form a CSA liquid; (ii) mixing the CSA liquid with a polymer adhesive composition, wherein the polymer adhesive composition comprises polyacrylate (e.g., pressure-sensitive polyacrylate), to form a CSA adhesive mixture; (iii) spreading the CSA adhesive mixture onto a releasable substrate; and (iv) allowing the CSA adhesive mixture to dry or at least partially solidify to form a polymer adhesive incorporating the one or more CSA compounds. The method can further comprise adding a backing substrate (e.g., according to any of the backing substrate examples disclosed herein) to an upper surface of the polymer adhesive after allowing the CSA adhesive mixture to dry.

The one or more CSA compounds when making a polyacrylate-based adhesive medical film can be included in an amount such that the CSA liquid includes the one or more CSA compounds at a concentration of about 20% (w/v) to about 50% (w/v), or about 25% (w/v) to about 45% (w/v), or about 30% (w/v) to about 40% (w/v), or about 35% (w/v), or a concentration within a range with endpoints selected from any two of the foregoing values.

After mixing of the CSA liquid with the polymer adhesive composition to form the CSA adhesive mixture, the CSA adhesive mixture can comprise a weight ratio of polyacrylate solids to the one or more CSA compounds of about 1.5 to about 3.5, such as about 2 to about 3, such as about 2.5, or a ratio within a range with endpoints selected from any two of the foregoing values.

The CSA adhesive mixture can be spread to a thickness of about 100 μm to about 350 μm, or such as about 150 μm to about 250 μm, or such as about 200 μm, or a thickness within a range with endpoints selected from any two of the foregoing values. While other thicknesses are possible, the foregoing were found to result, after drying, in the end thicknesses disclosed herein that were found to be effective.

In another embodiment, a silicone-based adhesive medical film can be manufactured by the steps of: (i) mixing one or more CSA compounds with an organic alcohol to form a CSA liquid; (ii) soaking a silicone film material in the CSA liquid such that the silicone film material swells and takes up at least a portion of the one or more CSA compounds of the CSA liquid; and (iii) optionally, sonicating the CSA liquid while the silicone film material is in the CSA liquid.

The one or more CSA compounds when making a silicone-based adhesive medical film can be included in an amount such that the CSA liquid includes the one or more CSA compounds at a concentration of about 0.5% (w/v) to about 5% (w/v), or such as about 0.75% (w/v) to about 2.5% (w/v), or such as about 1% (w/v), or within a range with endpoints selected from any two of the foregoing values.

### V. Antimicrobial Efficacy

The disclosed adhesive medical films can provide broad-spectrum antimicrobial activity against gram-positive bacteria (e.g., MRSA), gram-negative bacteria (e.g., PA), and pathogenic fungi (e.g., CA). As demonstrated in the disclosed Examples, the adhesive medical films can provide at least a 3-log reduction in colonization of gram-positive bacteria, gram-negative bacteria, and pathogenic fungi following 24 hours of exposure of these microbes to the polymer adhesive, as compared to the same adhesive medical film without incorporated CSA compounds.

As further demonstrated herein, the adhesive medical films can reduce growth of gram-positive bacteria, gram-negative bacteria, and pathogenic fungi, when continuously exposed thereto, for at least 6 days, as compared to the same adhesive medical film without incorporated CSA compounds. While this may be sufficient for many applications, other embodiments can provide further sustained antimicrobial activity. For example, where a CSA compound with non-hydrolysable linkages (e.g., CSA-131) is utilized, reductions in microbial growth can extend to about 68 days against MRSA, about 45 days against PA, and about 57 days against CA. Practical reductions in microbial growth are likely even greater considering that test conditions were intentionally designed to be conservative.

### VI. Working Examples

### 1. Results

### Development of Antimicrobial Adhesives:

One adhesive that was used to make an antimicrobial medical film was Aroset 700 Gentle, a type of pressure-sensitive polyacrylate adhesive. Aroset 700 Gentle formed homogeneous solutions with concentrated solutions of the tested ceragenins in ethanol. After removal of the ethanol, the resulting ceragenin-impregnated polyacrylate adhesive remained stable with desirable adhesive properties even with relatively large percentages of ceragenins.

To incorporate the ceragenin into the polyacrylate adhesive, the ceragenin was first dissolved in ethanol at a high concentration (e.g., about 35% (w/v)). The ethanol solution containing either CSA-44 or CSA-131 was then mixed with the polyacrylate adhesive, in a three-to-one ratio, to give a homogenous solution. This solution was spread to a thickness of 200 μm on capped “release paper.” The adhesive coating was allowed to thoroughly dry before a carrier substrate was added on top of the adhesive layer with gentle pressure. The carrier substrate used in this example was a polyamide/elastane pad acquired from Mederma. The combination of the adhesive on the substrate gave coated materials POLYA44 and POLYA131 (with POLYAC as the materials containing the adhesive without ceragenin).

Also explored was the use of a ceragenin in adhesive silicone scar tape, which is used on postoperative wounds to reduce infection as well as to reduce the production of scars. Colonization of scar tape has previously been reported as a risk factor in Healthcare-associated infections (HAIs) that can negatively impact patient outcomes. Silicone scar tapes are typically made with one face of the tape comprised of capped silicone, with the opposite side presenting uncapped silicone. The uncapped silicone has adhesive properties, and no additional adhesive is required to secure the tape to dermal or other soft tissue. Thus, to incorporate a CSA (ceragenin), a coating was not required. Rather, the CSA (ceragenin) was infused directly into the silicone. Silicone swells in the presence of organic alcohols, and exposure of scar tape to CSA-131 in isopropyl alcohol (IPA) resulted in infusion of the CSA (ceragenin) into the silicone tape. To accelerate this process, it was found that use of intense sonication resulted in greater amounts of the CSA (ceragenin) being incorporated into the silicone material. After exposure to CSA-131 in isopropyl alcohol and horn sonication, samples were allowed to dry, which allowed the silicone to resume its original shape, trapping the CSA (ceragenin) within the silicone. Samples were then rinsed with isopropyl alcohol to remove residual CSA-131 on the surface. Optimization of this method gave a material termed SCART131, whereas the untreated scar tape was designated scar tape control (SCARTC).

### Antimicrobial Properties of Adhesive Materials:

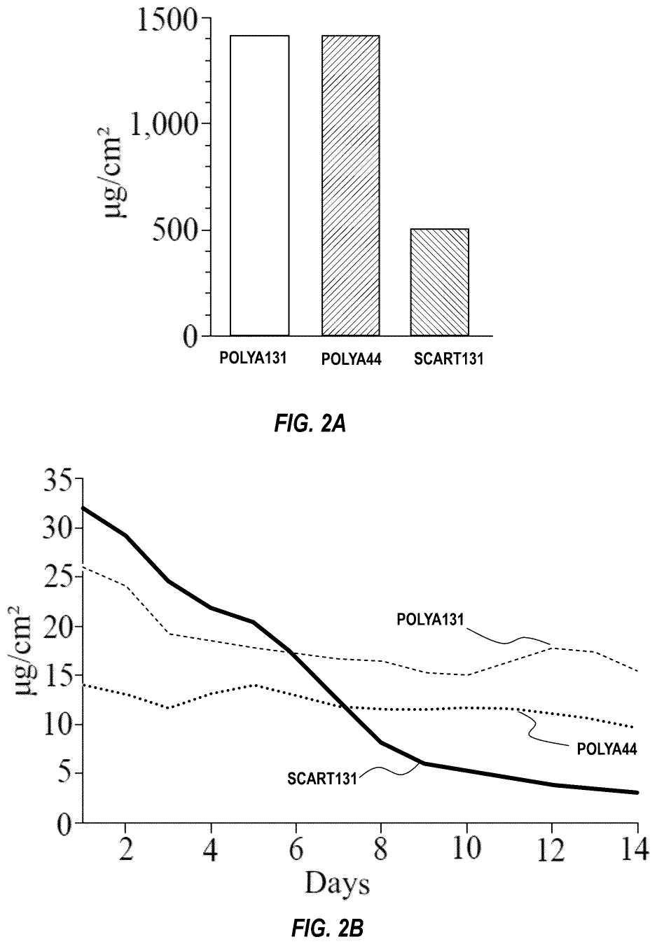

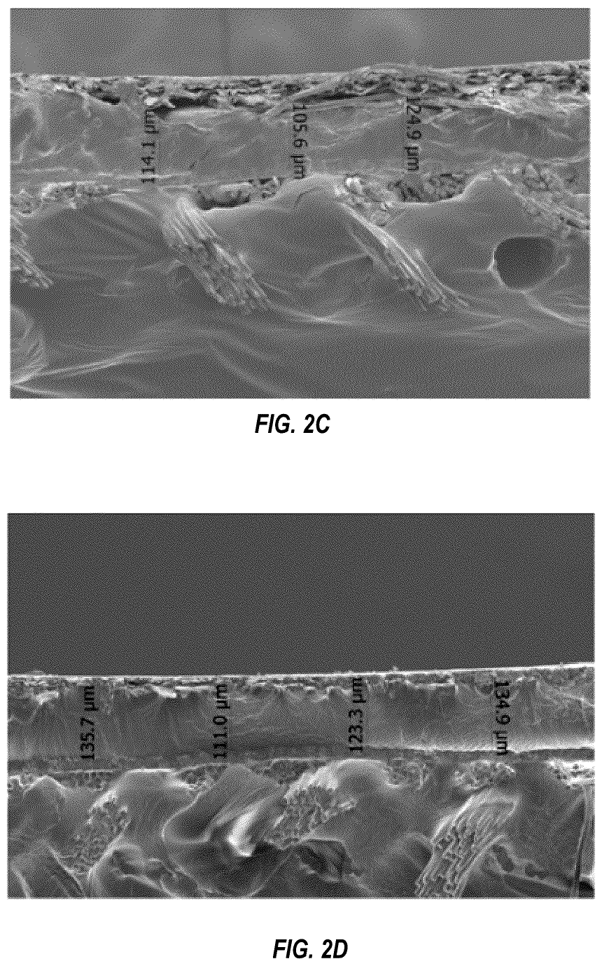

To measure the total amounts of ceragenin in the adhesives and scar tape, sections of adhesive-coated materials or scar tape were soaked in a mixture of isopropanol and dilute aqueous acid to allow complete release of CSA-44 or CSA-131 into the solution. As shown in FIG. 2A, both POLYA44 and POLYA131 contained approximately 1,400 μg/cm² of the CSAs (ceragenins), while SCART131 had a total CSA-131 extraction of about 500 μg/cm², which was significantly less than the polyacrylate adhesives. This lower amount was because the CSA (ceragenin) was infused into silicone rather than incorporated into a coating. Scar tape is typically used for relatively short timespans, so this lower amount was expected to be sufficient for most applications. Nevertheless, increased concentrations ceragenins can be infused into the silicone scar tape if desired (e.g., by multiple exposures and/or using more highly concentrated solutions).

To verify that the ceragenin reservoirs would be accessible and released in a controlled manner, elution assays were run into phosphate-buffered saline (PBS). Samples were immersed in PBS and incubated at 37° C. for 24 hours. Samples were moved to fresh PBS, and the process was repeated. Amounts of CSAs (ceragenins) that eluted daily are plotted in FIG. 2B as the amount released per square centimeter. SCART131 and POLYA131 gave the highest elution on the first day, which may be important in eliminating existing microbes at the sites of application. Notably, the CSAs (ceragenins) continued to controllably elute for more than 14 days, which was expected to provide a defense against the introduction of new microbes. POLYA44 showed a near zero-order release over the 14-day evaluation period.

The polyacrylate adhesives were also investigated for uniformity of the coating and to confirm coating thickness through SEM images of POLYA44 and POLYA131 (FIGS. 2C and 2D). To obtain the desired images, coated materials were frozen and then broken sharply. Though the sample of POLYA131 displayed some artifacts of the cracking process used to prepare the sample, such as some degree of delamination and loss of integrity in the release paper, there were no visible salt crystals or bubbles in either sample. The coatings appeared relatively uniform with a thickness of about 120 μm. Thus, the applied coatings, when dried, were reduced in thickness by about 40% compared to their initial applied thickness of 200 μm.

The primary objective was to generate adhesive materials with broad-spectrum antimicrobial properties, including activity against representative fungi and Gram-positive and Gram-negative bacteria. MRSA (ATCC BAA-41) was chosen as a representative Gram-positive organism, as it is a common pathogen in nosocomial infections. Multi-drug resistant PA (ATCC 47085) was chosen as the Gram-negative bacterium and CA (ATCC 90028) as a representative fungal pathogen commonly found in HAIs.

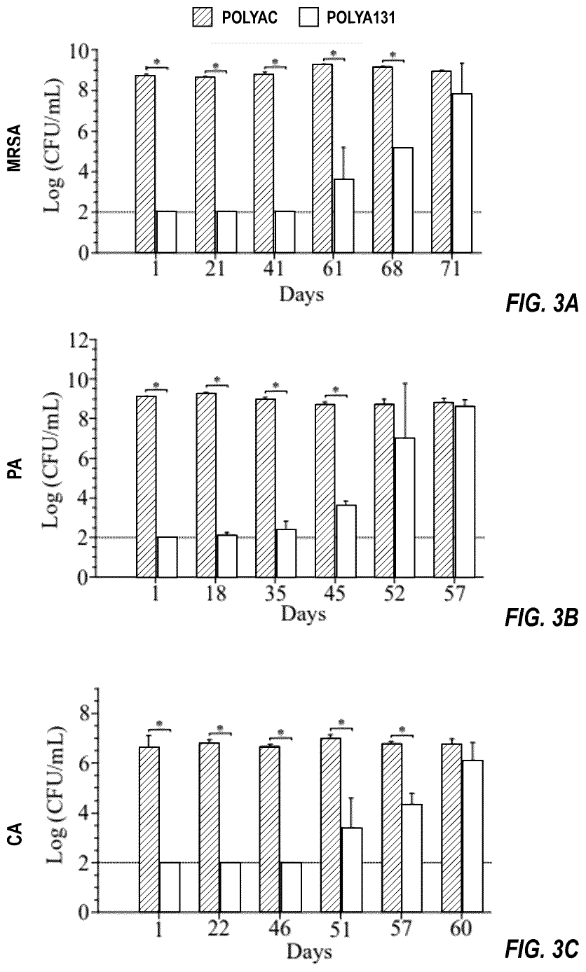

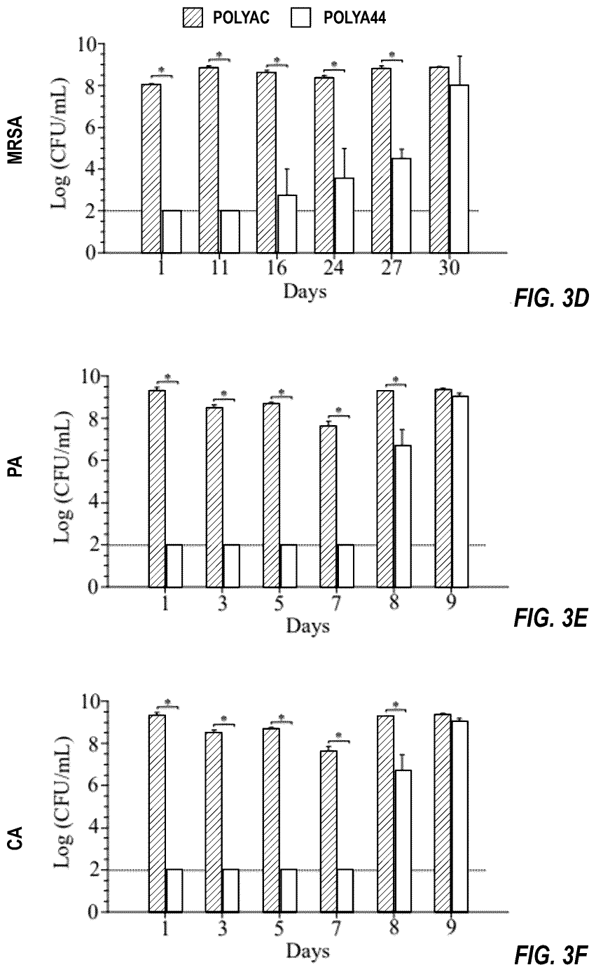

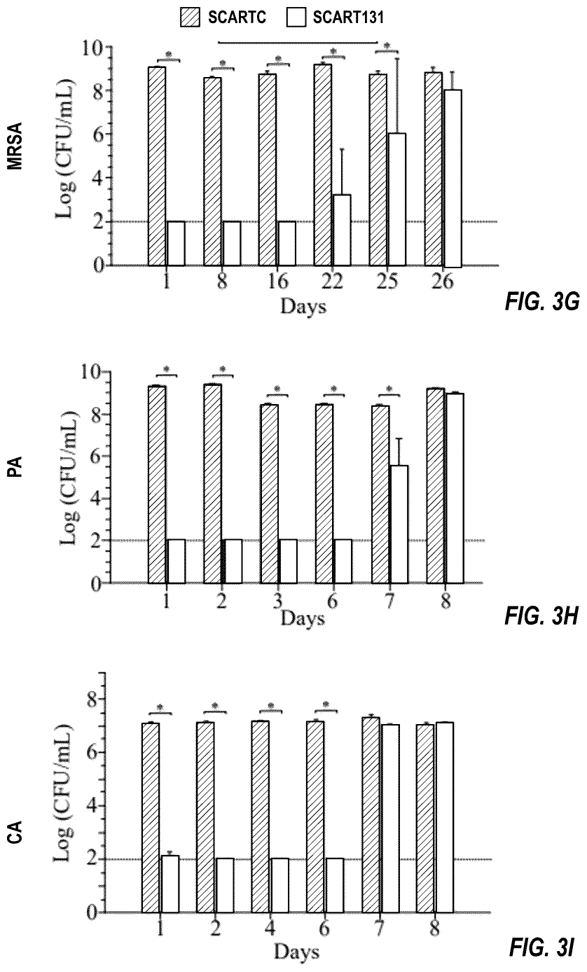

Adhesives POLYA131, POLYA44, and scar tape SCART131 were challenged against all three pathogens to determine how long the eluted antimicrobials would control fungal and bacterial growth. Samples were challenged daily with the indicated organisms with daily exchange of growth medium. FIGS. 3A-3C show the daily growth results of MRSA, PA, and CA, respectively, in the presence of POLYA131. FIGS. 3D-3F show the daily growth results of MRSA, PA, and CA, respectively, in the presence of POLYA44. FIGS. 3G-3I show the daily growth results of MRSA, PA, and CA, respectively, in the presence of SCART131.

Both the polyacrylate adhesives and the adhesive silicone performed best against MRSA, with POLYA131, POLYA44, and SCART131 showing significant reductions in bacterial growth, relative to controls, for 68, 27, and 25 days, respectively. Against PA, significant bacterial growth reduction was observed for 45, 8, and 7 days, respectively. Significant reduction in fungal growth was observed for 57, 12, and 6 days, respectively, with CA. Loss of activity against PA and CA with POLYA44 and SCART131 on days 6-8 is consistent with lower amounts of ceragenins CSA-44 and CSA-131 eluting on these days. Correlation of the amounts of the ceragenin released from the POLYA44 and SCART131 with antimicrobial activity provides information about the absolute amounts of the CSAs (ceragenins) required to eliminate the microbial inocula. Notably, CSA-131 release from POLYA131 remained higher than ceragenin release from the other materials. Consequently, this material demonstrated the longest-lasting antimicrobial activity. In addition, CSA-131 was found to be active at lower concentrations than CSA-44 against the organisms used in the evaluation of the adhesive materials, and the differences in duration of activity may be influenced by the native activity of the CSA (ceragenin) in question. Overall, a reduction of growth for all tested strains for at least six days is a promising benchmark, and a reduction of MRSA for three weeks is particularly notable and beneficial, given its prevalence in HAIs.

### Surface Sterilization:

To test how the adhesives performed in disinfecting a surface, antimicrobial assays were performed on agar. Inocula for each pathogen were spread across fungal or bacterial culture plates to generate a microbial lawn. Adhesive materials were placed on top of the agar and the plates were incubated overnight. The agar surface underneath the adhesive materials was collected for quantification of surviving pathogens.

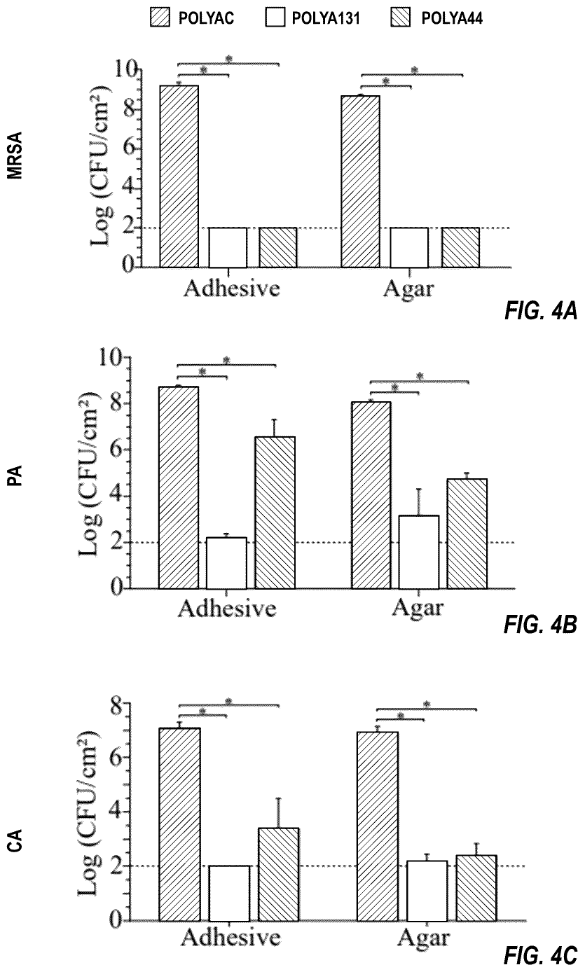

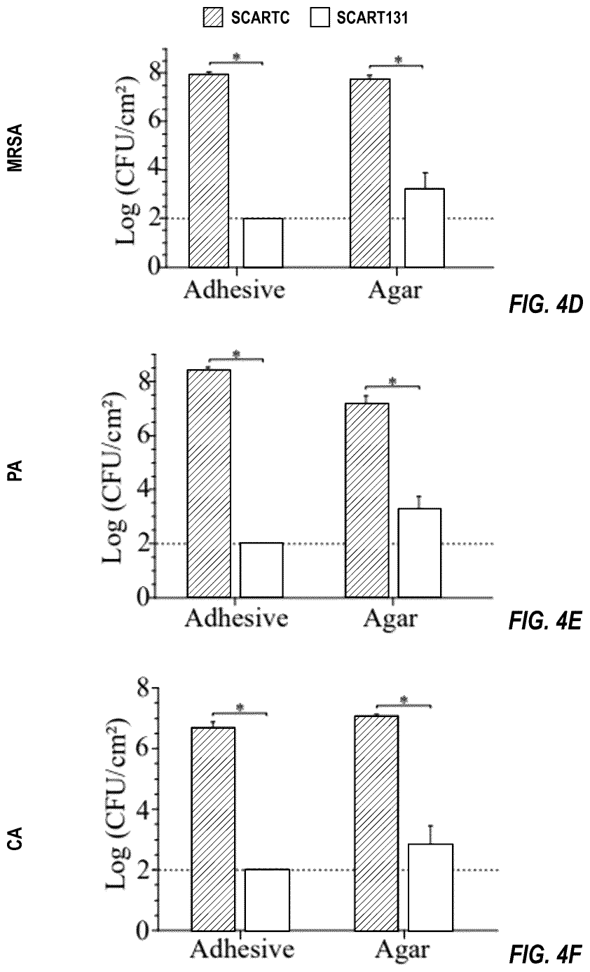

As shown in FIGS. 4A-4F, all adhesive materials significantly reduced the growth of all pathogens. Consistent with the results in aqueous media, the POLYA44 and POLYA131 adhesive materials performed best against MRSA, reducing surviving cells to the limit of detection. With PA and CA, growth was reduced by more than three logs by both POLYA44 and POLYA131 relative to controls. Similarly, SCART131 gave a greater than three-log reduction of all pathogens relative to controls. Due to the possibility that the adhesive could remove surviving cells, the removed adhesives were also evaluated for the presence of bacteria or fungi. As shown, all adhesives remained free of MRSA contamination and had reduced PA and CA contamination by more than four logs. These results suggest that the CSA (ceragenin)-containing adhesive materials may prevent cross-contamination so as to reduce or eliminate HAIs.

### Adhesive Strength Testing:

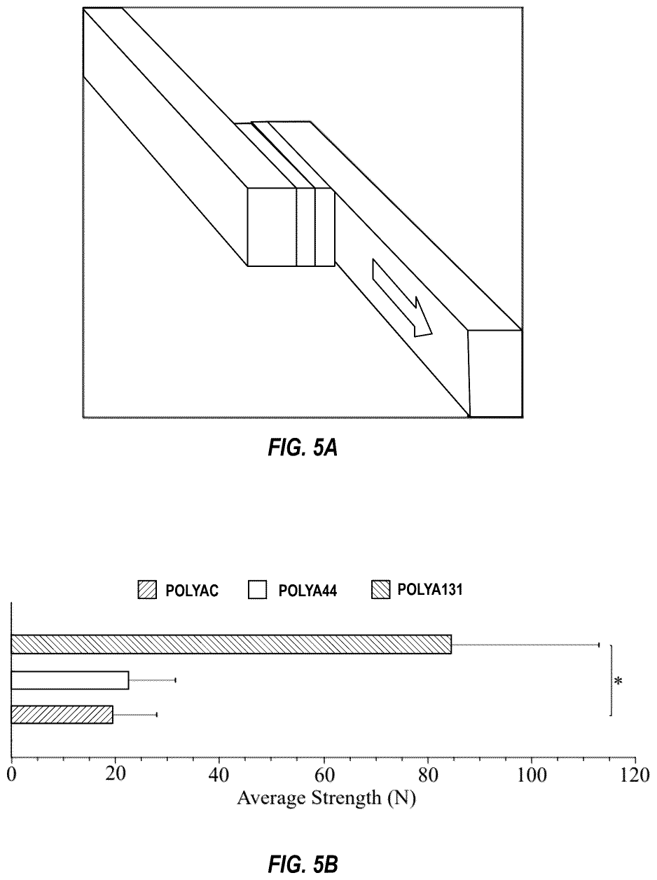

To assess the effects of CSA-44 and CSA-131 on the shear strength of adhesiveness, a lap shear test was performed (FIG. 5A). In this assay, the force required to cause failure of the adhesive was measured. With the parent adhesive (POLYAC), the force required was 20 N (FIG. 5B). A comparable, but slightly higher, force was required for POLYA44 (22.5 N). Surprisingly, the presence of CSA-131 in POLYA131 increased the adhesive strength of the adhesive, requiring 84.5 N to cause failure. Thus, there was only about a 12% increase between the adhesive with CSA-44 and that without any ceragenin antimicrobial agent, which may not be that significant. Interestingly and unexpectedly, adding CSA-131 significantly increased the adhesive strength, demonstrating a potential improvement in preventing adhesive movement.

### Dermal Irritation Measurements:

The primary application of the adhesive materials is expected to be on skin, and to ensure that the adhesives containing CSAs (ceragenins) did not cause irritation, the biocompatibilities of adhesive materials POLYA44 and POLYA131 were measured. Application locations on New Zealand white rabbits were shaved, and POLYA44 and POLYA131 patches were applied. The standard protocol for skin irritation evaluation (protocol ANSI/AAMI/ISO 10993-23-2021), calls for a test material residence time of 4 hours. Because use of ceragenin-adhesive materials is expected to be for longer periods, materials were left in place for 24 hours. At 24, 48 and 72 hours, sites for POLYA44 and POLYA131 application were evaluated for erythema and edema caused by the materials using scoring of 0-4: ranging from no erythema/edema (0) to severe erythema/edema (4). Both adhesive materials were characterized as non-irritating with scores of 0. The amount of CSA-131 in the scar tape was less than half than that found in POLYA131, and the initial release rates of CSA-131 from POLYA131 and SCART131 were comparable; therefore, SCART131 is also likely to be non-irritating.

### 2. Materials & Methods

### Preparation of Antimicrobial Polyacrylate Adhesives:

Aroset™ Gentle 700 pressure-sensitive adhesive was purchased from Ashland (Wilmington, USA) at 45% solids (w/w). Solutions of the HCl salts of CSA-44 and CSA-131 were prepared in ethanol at a concentration of 35% (w/v). The prepared ceragenin solutions were added to the Aroset Gentle 700 at in a 1:2 ratio (v/w). The ceragenin solutions and adhesive were mixed for 1 hour until visibly homogenous. The mixtures were poured onto silicone release paper and spread using a Gardco® Universal Blade Film Applicator (Gardco, Columbia USA) set to 200 μm to create a uniform layer. All adhesives were dried for 72 hours within a SafeAire fume hood (Fisher Hamilton Scientific, Two Rivers, USA) at room temperature to allow volatile substances to evaporate. Polyester cloth (VWR International, Radnor, USA) was used as the substrate pad for broth antimicrobial testing, and a polyamide/elastane blend (Mederma, Raleigh USA) was used as a substrate for imaging and surface disinfection assays. The cloth substrates were sterilized by immersion in isopropanol for 60 seconds, followed by immersion in hexanes for 30 seconds. The cleansed substrate was allowed to dry for 24 hours at room temperature before being placed on top of the adhesive with light pressure. Sections for extraction, elution and broth antimicrobial evaluation were formed using a 10 mm square metal die.

### Preparation of Antimicrobial Scar Tape:

To prepare the ceragenin-containing scar tape, sections of Mederma Scar Sheets were punched in 1 cm squares using a template metal die. After removing the release paper covering the adhesive face, squares were immersed in a 1% CSA-131 (w/v) isopropyl alcohol solution. The solution and scar tape were sonicated with a Branson Digital Sonifier SFX 550 horn sonicator (Ferguson, USA) for 15 minutes at 275 watts. After sonication, scar tape samples were removed from the solution and air-dried for 12 hours with the uncapped side facing up. Each scar tape square was rinsed in 10 mL of isopropanol for 2 minutes to remove surface CSA (ceragenin) molecules. The scar tape was then dried again for 12 hours, and release paper was reapplied to the adhesive surface.

### Total Extraction of Ceragenins:

The total amount of CSA 131 and CSA 44 loaded into the adhesive materials was determined by immersing 10 mm adhesive squares, with release paper removed, into a solution of 20% 1 N aqueous HCL in isopropyl alcohol at a temperature of 60° C. The extraction solution was collected and replaced regularly, with the first three fractions collected every 8 hours, followed by four fractions collected after 24 hour intervals at 37° C. After each fraction was collected, the concentrations of CSA-131 and CSA-44 were determined via mass spectrometry using mass-labeled internal standards (CSA-131D₂₅ and CSA-44D₂). Calculated concentrations of the seven fractions were summed for each sample set. Sample extractions were run in triplicate with error bars representing standard deviations among extraction totals.

### Quantification of Ceragenin Elution:

Square sections (10 mm) of adhesive materials containing CSA-131 and CSA-44 were prepared as described above. Release paper was removed, and samples were placed in 5 mL conical tubes and immersed in PBS (1 mL). The conical tubes were placed in an incubator at 37° C. for 24 hours. After the incubation period, the PBS solution was removed, and equal parts of the collected solution and a deuterated internal standard were mixed. The mixture was evaluated via mass spectrometry to determine the concentration of ceragenin eluted over the 24 hour period of incubation. At 24 hour intervals, sections were rinsed once with PBS and submerged in fresh PBS. The process was repeated daily for 2 weeks.

### Scanning Electron Microscopy:

Adhesive material strips were immersed in liquid nitrogen to allow fracture. After 15 minutes, strips were removed and snapped by bending. The samples were mounted on SEM pin stubs and sputter-coated with approximately 20 nm of a gold-palladium alloy using a Quorum Q 150T ES (Electron Microscopy Sciences, Hatfield, PA, USA). The SEM stub was loaded onto a 45° chamfer in the mounting stage. The stage was further tilted by an additional 45° to allow normal imaging of the snapped cross-section of the adhesive strip. Images and measurements of the adhesive cross sections were obtained using an Apreo C microscope (Thermo Fisher Scientific, Waltham, MA, USA).

### Microbial Cultures:

Bacterial and fungal cultures were prepared from fresh colonies placed in media and incubated overnight at 37° C. PA (PA01, ATCC 47085) and MRSA (ATCC BAA-41) were cultured in trypticase soy broth (TSB). CA (ATCC 90028) was cultured in Emmons Modified Sabouraud Dextrose Broth (EMSDB). The cultures were centrifuged and rinsed three times in phosphate-buffered saline (PBS) and resuspended in PBS. Optical density (OD) readings were performed at 600 nm on a Genesys 30 spectrophotometer to determine cell density. Cell suspensions were diluted to form inocula of 10³ CFU/mL in 10% TSB in PBS and 10³ CFU/mL in 10% EMSDB in PBS for bacteria and fungi, respectively.

### Evaluation of Antimicrobial Properties of Adhesives:

Adhesive materials were prepared as described above, and a 10 mm square metal cutting die (Walfront, Lewes, USA) was used to generate identical sections for microbial challenge. Release paper was removed, and adhesive material sections were placed individually in wells of a 12-well plate, immersed in 1 mL of inocula and incubated for 24 hours. Microbial growth was quantified by removing 20 μL aliquots and adding them to Dey-Engley Neutralizing Broth (Sigma-Aldrich, St. Louis, MO, USA). The resulting suspensions were serially diluted in a 96 well plate and 100 μL aliquots of the resulting suspensions were spread on nutrient agar. Bacterial plates were incubated for 24 hours, and fungal plates were incubated for 48 hours before colonies were counted. After sampling, the remaining growth medium was removed from the adhesive sections. The sections were washed three times with PBS and transferred to new 12-well plates, immersed in nutrient media and inoculated with fresh bacterial or fungal culture. Samples were incubated for an additional 24 hours. This process was repeated every 24 hours. The study was run in triplicate and growth was measured daily until statistical significance was lost as determined by student's t-test (p<0.05).

### Surface Sterilization:

Circular samples with a radius of 10 mm of each test adhesive material were generated using a metal cutting die. A lawn of the indicated pathogen was generated by streaking agar plates with a cotton swabbed dipped in a 5×10⁷ CFU/mL suspension. Release paper was removed from the adhesive samples, which were then placed onto the prepared plates. Plates were allowed to incubate for 24 hours. After the incubation period, the adhesive materials were removed and placed into neutralizing broth, sonicated for 10 minutes and vortexed for 1 minute. The resulting suspension was serially diluted, spread on agar plates, and incubated. The agar under the adhesive was also sampled, with a 10 mm square section being removed, placed in neutralizing broth and sonicated for 10 minutes to release surviving cells. The resulting suspension was serially diluted, spread on agar plates and incubated. Colonies were quantified after 24 hours for bacteria and after 48 hours for fungi.

### Single-Lap Shear Test:

A single-lap shear test is a common method for evaluating adhesive strength. In this test, a single layer of adhesive is sandwiched between two metal or polymer substrates, and a shear load is applied parallel to the plane of the substrates (FIG. 5A). The test provides a load-displacement curve to determine the shear strength of the adhesive bond following the ASTM D4896-01 standard. The adhesive strength without any additive was evaluated against the adhesive strength with addition of the two different ceragenins. A 50 mm×25.7 mm×0.08 mm layer of adhesive was placed between two 73 mm×25.7 mm×6.5 mm high density polyethylene (HDPE) substrates and pressed for 2 hours under 1.5 lbs. (0.68 kg) of weight. The HDPE substrates were then separated at a rate of 5 mm/min using a 3345 series Instron tensile strength tester to measure the load and extension. The maximum load values for the samples were averaged to get a representative strength of adhesion for each adhesive formulation.

### Dermal Irritation Study in Rabbits:

Skin irritation potential of adhesive patches containing either CSA-44 (POLYA44) or CSA-131 (POLYA131) was evaluated following protocol ANSI/AAMI/ISO 10993-23-2021, with the following modifications: adhesive patches remained in place for 24 hours (rather than 4 hours as in the protocol), and patch sizes were 2×2 cm (rather than 2.5×2.5 cm in the protocol). The study was performed at Geneva Laboratories (Elkhorn, WI) and with New Zealand white rabbits (Oryctolagus cuniculus) under IACUC approval from a standing committee at Geneva Laboratories. Briefly: the backs of three animals were clipped free of hair to expose 15×15 cm of skin. Two control patches, two containing CSA-44 and two containing CSA-131 were applied to each animal. To ensure that patches remained in place, patches were covered with a self-adhering wrap and covered with a cloth stocking. After 24 hours, coverings and patches were removed and the covered skin was evaluated for erythema and edema using scoring of 0-4, ranging from no erythema/edema (0) to severe erythema/edema (4). Evaluation was performed by a trained pathologist at 24, 48 and 72 hours. Skin under the patches, both control and test, were scored as 0 at all time points. To validate the protocol, a positive validation test is performed at Geneva Laboratories every six months using sodium dodecyl sulfate in petroleum jelly, which scores as a “moderate” irritant.

### VII. Additional Terms & Definitions

While certain embodiments of the present disclosure have been described in detail, with reference to specific configurations, parameters, components, elements, etcetera, the descriptions are illustrative and are not to be construed as limiting the scope of the claimed invention.

For any given element of component of a described embodiment, any of the possible alternatives listed for that element or component may generally be used individually or in combination with one another, unless implicitly or explicitly stated otherwise.

The various features of a given embodiment can be combined with and/or incorporated into other embodiments disclosed herein. Thus, disclosure of certain features relative to a specific embodiment of the present disclosure should not be construed as limiting application or inclusion of said features to the specific embodiment. Rather, it will be appreciated that other embodiments can also include such features.

Unless otherwise indicated, numbers expressing quantities, constituents, distances, or other measurements used in the specification and claims are to be understood as optionally being modified by the term “about.” When the terms “about,” “approximately,” “substantially,” or the like are used in conjunction with a stated amount, value, or condition, it may be taken to mean an amount, value or condition that deviates by less than 10%, less than 5%, less than 1%, less than 0.1%, or less than 0.01% of the stated amount, value, or condition. Each numerical parameter should be construed in light of the number of reported significant digits and by applying ordinary rounding techniques.

Any headings and subheadings used herein are for organizational purposes only and are not meant to be used to limit the scope of the description or the claims.

As used in this specification and the appended claims, the singular forms “a,” “an” and “the” do not exclude plural referents unless the context clearly dictates otherwise. Thus, for example, an embodiment referencing a singular referent (e.g., “CSA compound”) may also include two or more such referents.

The embodiments disclosed herein should be understood as comprising/including disclosed components, and may therefore include additional components not specifically described. Optionally, the embodiments disclosed herein are essentially free or completely free of components that are not specifically described. That is, non-disclosed components may optionally be completely omitted or essentially omitted from the disclosed embodiments. For example, polymers and/or coating components not disclosed herein may optionally be completely omitted or essentially omitted.

An embodiment that “essentially omits” or is “essentially free of” a component may include trace amounts and/or non-functional amounts of the component. For example, an “essentially omitted” component may be included in an amount no more than 1%, no more than 0.1%, or no more than 0.01% by total weight of the relevant composition (e.g. by total weight of the coating composition from which a coating layer is formed).

A composition that “completely omits” or is “completely free of” a component does not include a detectable amount of the component (i.e., does not include an amount above any inherent background signal associated with the testing instrument) when analyzed using standard compositional analysis techniques such as, for example, chromatographic techniques (e.g., thin-layer chromatography (TLC), gas chromatography (GC), liquid chromatography (LC)), or spectroscopy techniques (e.g., Fourier transform infrared (FTIR) spectroscopy).

### VIII. Additional CSA Compound Details

Exemplary CSA compounds and methods for their manufacture are described in U.S. Pat. Nos. 6,350,738, 6,486,148, 6,767,904, 7,598,234, 7,754,705, 8,691,252, 8,975,310, 9,434,759, 9,527,883, 9,943,614, 10,155,788, 10,227,376, 10,370,403, 10,626,139, 11,286,276, 12,030,912, and 12,215,126, and U.S. Pat. Pub. Nos. 2016/0311850 and 2025/0073248, which are incorporated herein by reference. The skilled artisan will recognize the compounds within the generic formulae set forth herein and understand their preparation in view of the references cited herein and the Examples contained therein.

CSA compounds can have a structure of Formula I, Formula II, Formula III, and/or Formula IV. Formula III differs from Formula I and II by omitting R₁₅ and the ring carbon to which it is attached. Formula IV more particularly defines Formula III with respect to stereochemistry and R groups for all but R₃, R₇, R₁₂, and R₁₈.

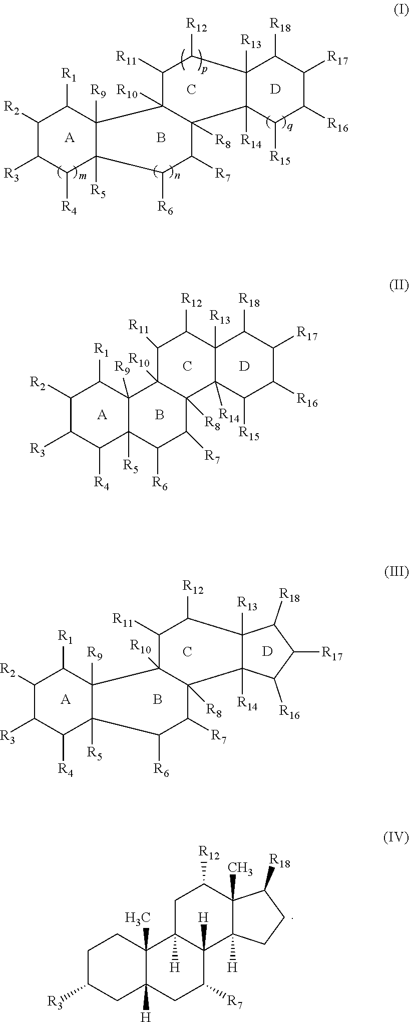

In embodiments of Formulas I, II, III, and IV, at least two of R₃, R₇, and R₁₂ may independently include a cationic moiety (e.g., amino or guanidino groups) bonded to the steroid backbone structure via a hydrolysable or non-hydrolysable linkage. For the embodiments of the present disclosure, the linkage is preferably hydrolysable but stable under conditions of sterilization and storage, and hydrolysable under physiological conditions. Such cationic functional groups (e.g., amino or guanidino groups) may be separated from the backbone by at least one, two, three, four or more atoms.

A tail moiety may be attached to the sterol backbone at R₁₈, may have variable chain length or size, and may be charged, uncharged, polar, non-polar, hydrophobic, or amphipathic. The tail moiety may be used to select the hydrophobicity/hydrophilicity of the ceragenin compound. CSA compounds having different degrees of hydrophobicity/hydrophilicity may have different rates of uptake into different target microbes.

The “R” groups described herein, unless specified otherwise, may be substituted or unsubstituted.

With respect to CSA compounds of Formulas I, II, and III (and where not already specified with respect to Formula IV):
each of fused rings A, B, C, and D may be independently saturated, or may be fully or partially unsaturated, provided that at least two of A, B, C, and D is saturated, wherein rings A, B, C, and D form a ring system. Other ring systems can also be used, e.g., 5-member fused rings and/or compounds with backbones having a combination of 5- and 6-membered rings;
R₁ through R₁₈ are independently selected from the group consisting of hydrogen, hydroxyl, alkyl, hydroxyalkyl, alkyloxyalkyl, alkylcarboxyalkyl, terpenylcarboxyalkyl, terpenylcarbonyloxyalkyl, terpenylamidoalkyl, terpenylaminoalkyl, terpenyloxyoalkyl, alkylaminoalkyl, alkylamino-alkylamino, alkylaminoalkylaminoalkylamino, aminoalkyl, aryl, arylaminoalkyl, haloalkyl, alkenyl, alkynyl, oxo, linking group attached to a second steroid, aminoalkylurethanyl, aminoalkenylurethanyl, aminoalkynylurethanyl, aminoarylurethanyl, aminoalkyloxy, aminoalkylcarboxy, aminoalkyloxyalkyl, aminoalkylaminocarbonyl, aminoalkylcarboxamido, di(alkyl)aminoalkyl, H₂N—HC(Q₅)-(C═O)—O—, H₂N—HC(Q₅)-(C═O)—NH—, azidoalkyloxy, cyanoalkyloxy, P.G.-HN—HC(Q₅)-(C═O)—O—, guanidinoalkyloxy, quaternary ammonium alkylcarboxy, and guanidinoalkyl carboxy, where Q₅ is a side chain of any amino acid (including a side chain of glycine, i.e., H), and P.G. is an amino protecting group; and
R₅, R₈, R₉, R₁₀, R₁₃, R₁₄ and R₁₇ are independently deleted when one of rings A, B, C, or D is unsaturated so as to complete the valency of the carbon atom at that site, provided that at least one, and sometimes two, three, or four, of R₁₋₄, R₆, R₇, R₁₁, R₁₂, R₁₅, R₁₆, R₁₇, and R₁₈ are independently selected from the group consisting of aminoalkyl, aminoalkyloxy, aminoalkylcarboxyalkyl, alkylaminoalkyl, alkylamino-alkylamino, alkylaminoalkylaminoalkylamino, aminoalkylcarboxy, aryl-aminoalkyl, aminoalkyloxyamino, alkylaminocarbonyl, aminoalkylaminocarbonyl, aminoalkyl-carboxyamido, di(alkyl)aminoalkyl, aminoalkylurethanyl, aminoalkenyl-urethanyl, aminoalkynylurethanyl, aminoarylurethanyl, H₂N—HC(Q₅)-C(O)—O—, H₂N—HC(Q₅)-C(O)—N(H)—, azidoalkyloxy, cyanoalkyloxy, P.G.-HN—HC(Q₅)-C(O)—O—, guanidinoalkyloxy, quaternary ammonium alkylcarboxy, and guanidinoalkylcarboxy.

In embodiments, R₁ through R₄, R₆, R₇, R₁₁, R₁₂, R₁₅, R₁₆, and R₁₈ are independently selected from the group consisting of hydrogen, hydroxyl, substituted or unsubstituted (C₁-C₂₂)alkyl, substituted or unsubstituted (C₁-C₂₂)hydroxyalkyl, substituted or unsubstituted (C₁-C₂₂)alkyloxy-(C₁-C₂₂)alkyl, substituted or unsubstituted (C₁-C₂₂)alkylcarboxy-(C₁-C₂₂)alkyl, substituted or unsubstituted (C₅-C₂₅)terpenyl-carboxy-(C₁-C₂₂)alkyl, substituted or unsubstituted (C₅-C₂₅)terpenylcarbonyloxy-(C₁-C₂₂)alkyl, substituted or unsubstituted (C₅-C₂₅)terpenylcarboxamido-(C₁-C₂₂)alkyl, substituted or unsubstituted (C₅-C₂₅)terpenylamino-(C₁-C₂₂)alkyl, (C₅-C₂₅)terpenyloxyo-(C₁-C₂₂)alkyl, substituted or unsubstituted (C₁-C₂₂)alkylamino-(C₁-C₂₂)alkyl, substituted or unsubstituted (C₁-C₂₂)alkylamino-(C₁-C₂₂)alkylamino, substituted or unsubstituted (C₁-C₂₂)alkylamino-(C₁-C₂₂)alkylamino-(C₁-C₂₂)alkylamino, substituted or unsubsti-tuted (C₁-C₂₂)aminoalkyl, substituted or unsubstituted aryl, substituted or unsubstituted arylamino-(C₁-C₂₂)alkyl, substituted or unsubstituted (C₁-C₂₂)haloalkyl, substituted or unsubstituted (C₂-C₆)alkenyl, substituted or unsubstituted (C₂-C₆)alkynyl, oxo, linking group attached to a second steroid, substituted or unsubstituted (C₁-C₂₂)aminoalkylurethanyl, substituted or unsubstituted (C₂-C₂₂)aminoalkenylurethanyl, substituted or unsubstituted (C₂-C₂₂)aminoalkynylurethanyl, and substituted or unsubstituted aminoarylurethanyl, substituted or unsubstituted (C₁-C₂₂)aminoalkyloxy, substituted or unsubstituted (C₁-C₂₂)aminoalkylcarboxy, substituted or unsubstituted (C₁-C₂₂)aminoalkyloxy-(C₁-C₂₂)alkyl, substituted or unsubstituted (C₁-C₂₂)aminoalkyl-aminocarbonyl, substituted or unsubstituted (C₁-C₂₂)aminoalkylcarboxamido, substituted or unsubstituted di(C₁-C₂₂)alkylamino-(C₁-C₂₂)alkyl, H₂N—HC(Q₅)-(C═O)—O—, H₂N—HC(Q₅)-(C═O)—NH—, substituted or unsubstituted (C₁-C₂₂)azidoalkyloxy, substituted or unsubstituted (C₁-C₂₂) cyanoalkyloxy, P.G.-HN—HC(Q₅)-(C═O)—O—, substituted or unsubstituted (C₁-C₂₂) guanidinoalkyloxy, substituted or unsubstituted quaternary ammonium (C₁-C₂₂)alkylcarboxy, and substituted or unsubstituted (C₁-C₂₂) guanidinoalkyl carboxy, where Q₅ is a side chain of an amino acid (including a side chain of glycine, i.e., H), and P.G. is an amino protecting group; and
R₅, R₈, R₉, R₁₀, R₁₃, R₁₄ and R₁₇ are independently deleted when one of rings A, B, C, or D is unsaturated so as to complete the valency of the carbon atom at that site, or R₅, R₈, R₉, R₁₀, R₁₃, and R₁₄ are independently selected from the group consisting of hydrogen, hydroxyl, (C₁-C₂₂)alkyl, (C₁-C₂₂)hydroxyalkyl, (C₁-C₂₂)alkyloxy-(C₁-C₂₂)alkyl, (C₁-C₂₂) aminoalkyl, aryl, (C₁-C₂₂)haloalkyl, (C₂-C₆)alkenyl, (C₂-C₆)alkynyl, oxo, a linking group attached to a second steroid, (C₁-C₂₂)aminoalkyloxy, (C₁-C₂₂)aminoalkylcarboxy, (C₁-C₂₂)aminoalkylaminocarbonyl, di(C₁-C₂₂ alkyl)amino-(C₁-C₂₂)alkyl, H₂N—HC(Q₅)-C(O)—O—, H₂N—HC(Q₅)-C(O)—N(H)—, (C₁-C₂₂)azidoalkyloxy, (C₁-C₂₂) cyanoalkyloxy, P.G.-HN—HC(Q₅)-C(O)—O—, (C₁-C₂₂) guanidinoalkyloxy, and (C₁-C₂₂) guanidinoalkylcarboxy, where Q₅ is a side chain of an amino acid, and P.G. is an amino protecting group;
provided that at least two or three of R₁-4, R₆, R₇, R₁₁, R₁₂, R₁₅, R₁₆, R₁₇, and R₁₈ are independently selected from the group consisting of (C₁-C₂₂)aminoalkyl, (C₁-C₂₂)aminoalkyloxy, (C₁-C₂₂)alkylcarboxy-(C₁-C₂₂)alkyl, (C₁-C₂₂)alkylamino-(C₁-C₂₂)alkylamino, (C₁-C₂₂)alkylamino-(C₁-C₂₂)alkylamino-(C₁-C₂₂)alkylamino, (C₁-C₂₂)aminoalkylcarboxy, arylamino-(C₁-C₂₂)alkyl, (C₁-C₂₂)aminoalkyloxy (C₁-C₂₂)aminoalkylaminocarbonyl, (C₁-C₂₂)aminoalkylaminocarbonyl, (C₁-C₂₂)aminoalkyl-carboxyamido, quaternary ammonium (C₁-C₂₂)alkylcarboxy, di(C₁-C₂₂ alkyl)amino-(C₁-C₂₂)alkyl, (C₁-C₂₂)aminoalkylurethanyl, (C₂-C₂₂)aminoalkenylurethanyl, (C₂-C₂₂)amino-alkynylurethanyl, aminoarylurethanyl, H₂N—HC(Q₅)-C(O)—O—, H₂N—HC(Q₅)-C(O)—N(H)—, (C₁-C₂₂)azidoalkyloxy, (C₁-C₂₂) cyanoalkyloxy, P.G.-HN—HC(Q₅)-C(O)—O—, (C₁-C₂₂) guanidinoalkyloxy, and (C₁-C₂₂) guanidinoalkylcarboxy.

In embodiments, at least one of R₁-R₁₈, preferably R₁₈, can have the following bioresorbable mono- or diglyceride structure:

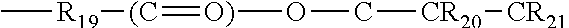

where R₁₉ is omitted or is selected from alkyl, alkenyl, alkynyl, and aryl, and R₂₀ and R₂₁ are independently selected from hydroxy and (C₂-C₂₂)alkylcarboxy, provided that at least one of R₂₀ or R₂₁ is (C₂-C₂₂)alkylcarboxy, the (C₂-C₂₂)alkylcarboxy preferably having an even number of carbons. The glyceride portion of foregoing structure forms bioresorbable glycerin and fatty acid(s) as degradation product (e.g., by hydrolysis of ester groups in the glyceride structure).

In some embodiments, R₁₈ can have the following bioresorbable mixed diglyceride structure:

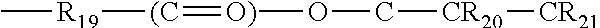

where R₁₉ is omitted or is selected from alkyl, alkenyl, alkynyl, and aryl, R₂₀ is a (C₂-C₂₂)alkylcarboxy, the (C₂-C₂₂)alkylcarboxy preferably having an even number of carbons, and R₂₁ can have the following aminoalkylcarboxy structure:

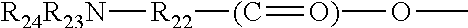

where R₂₂ is a substituted or unsubstituted alkyl and R₂₃ and R₂₄ are independently selected from hydrogen, alkyl, alkenyl, alkynyl, and aryl. R₂₂ is preferably an ester group of an amino acid, such as beta-alanine, which forms a bioresorbable amino acid (e.g., beta-alanine) as degradation product (e.g., by hydrolysis of the ester groups at the C24). The glyceride portion forms bioresorbable glycerin, a fatty acid, and an amino acid as degradation products (e.g., by hydrolysis of ester groups in the glyceride structure).

In some embodiments, at least one of R₁-R₁₈, preferably at least one of R₃, R₇ and R₁₂, can have the following bioresorbable aminoalkylcarboxy or aminoalkylcarboxamido structure:

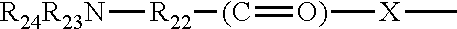

where R₂₂ is a substituted or unsubstituted alkyl, X is oxygen or nitrogen, and R₂₃ and R₂₄ are independently selected from hydrogen, alkyl, alkenyl, alkynyl, and aryl. At least one of R₃, R₇ and R₁₂, preferably two or three of R₃, R₇ and R₁₂, is/are an ester group of one or more amino acids, such as beta-alanine, which forms a bioresorbable amino acid (e.g., beta-alanine) as degradation product (e.g., by hydrolysis of the ester group(s) at the C3, C7 and/or C12 position(s)). Alternatively, the aminoalkyl portion of at least one of R₃, R₇ and R₁₂ can be attached to one or more of the C3, C7 and/or C12 positions of the sterol backbone (or elsewhere) by other linkages, such as amide or ether linkage.

In some embodiments, bioresorbable mono- and diglyceride CSA compounds can have a chiral center, such as in the glyceryl moiety, so as to form enantiomers that can be isolated rather than forming a racemic mixture. Unless otherwise specified, the examples of CSA compounds disclosed herein can be non-chiral, R- and S-enantiomers forming a racemic mixture, the R-enantiomer, or the S-enantiomer.

Non-limiting examples of bioresorbable monoglyceride CSA compounds that form bioresorbable degradation products by hydrolysis of ester groups are CSA-4108, CSA-4110 (racemic mixture), CSA-4110R (R-enantiomer), CSA-4110S (S-enantiomer), CSA-4112, CSA-4114, and salts thereof (see FIG. 1D). Non-limiting examples of bioresorbable diglyceride CSA compounds that form bioresorbable degradation products by hydrolysis of ester groups are CSA-4204, CSA-4206, CSA-4208, CSA-4210, and CSA-4310, and salts thereof (see FIG. 1D).

In embodiments, R₁, R₂, R₄, R₅, R₆, R₈, R₉, R₁₀, R₁₁, R₁₃, R₁₄, R₁₅, R₁₆, and R₁₇ are independently selected from the group consisting of hydrogen and unsubstituted (C₁-C₆)alkyl.

In embodiments, R₁, R₂, R₄, R₅, R₆, R₈, R₁₀, R₁₁, R₁₄, R₁₆, and R₁₇ are each hydrogen and Ry and R₁₃ are each methyl.

In embodiments, R₃, R₇, R₁₂, and R₁₈ are independently selected from the group consisting of hydrogen, (C₁-C₆)alkyl, (C₁-C₆)hydroxyalkyl, (C₁-C₁₆)alkyloxy-(C₁-C₅)alkyl, (C₁-C₁₆)alkylcarboxy-(C₁-C₅)alkyl, (C₁-C₁₆)alkylamino-(C₁-C₅)alkyl, (C₁-C₁₆)alkylamino-(C₁-C₅)alkylamino, (C₁-C₁₆)alkylamino-(C₁-C₁₆)alkylamino-(C₁-C₅)alkylamino, (C₅-C₂₅)terpenylcarboxy-(C₁-C₅)alkyl, (C₅-C₂₅)terpenylcarbonyloxy-(C₁-C₅)alkyl, (C₅-C₂₅)terpenylcarboxamido-(C₁-C₅)alkyl, (C₅-C₂₅)terpenylamino-(C₁-C₅)alkyl, (C₅-C₂₅)terpenyloxyo-(C₁-C₅)alkyl, (C₁-C₆)aminoalkylurethanyl, (C₂-C₆)aminoalkenylurethanyl, (C₂-C₆)aminoalkynylurethanyl, aminoarylurethanyl, (C₁-C₁₆)aminoalkyl, arylamino-(C₁-C₅)alkyl, (C₁-C₅)aminoalkyloxy, (C₁-C₁₆)aminoalkyl-oxy-(C₁-C₅)alkyl, (C₁-C₅)aminoalkylcarboxy, (C₁-C₅)aminoalkyl-aminocarbonyl, (C₁-C₅)aminoalkylcarbox-amido, di(C₁-C₈alkyl)amino-(C₁-C₅)alkyl, (C₁-C₅) guanidino-alkyloxy, quaternary ammonium (C₁-C₁₆)alkylcarboxy, and unsubstituted (C₁-C₁₆) guanidinoalkylcarboxy.

In embodiments, R₁, R₂, R₄, R₅, R₆, R₈, R₁₀, R₁₁, R₁₄, R₁₆, and R₁₇ are each hydrogen; and R₉ and R₁₃ are each methyl.

In embodiments, R₃, R₇, R₁₂, and R₁₈ are independently selected from the group consisting of aminoalkyloxy, aminoalkylcarboxy, alkylaminoalkyl, alkoxycarbonylalkyl, alkylcarbonylalkyl, di(alkyl)aminoalkyl, alkylcarboxyalkyl, hydroxyalkyl, terpenylcarboxyalkyl, terpenylcarbonyloxyalkyl, terpenylcarboxamido-alkyl, terpenylaminoalkyl, terpenyloxyoalkyl, aminoalkylurethanyl, aminoalkenylurethanyl, aminoalkynylurethanyl, and aminoarylurethanyl.

In embodiments, R₃, R₇, and R₁₂ are independently selected from the group consisting of aminoalkyloxy, aminoalkylcarboxy, aminoalkylurethanyl, aminoalkenyl-urethanyl, aminoalkynylurethanyl, and aminoarylurethanyl.

In embodiments, R₁₈ is selected from the group consisting of alkylaminoalkyl, alkoxycarbonylalkyl, alkylcarbonyloxyalkyl, alkylcarbonylalkyl, di(alkyl)aminoalkyl, alkylcarboxyalkyl, hydroxyalkyl, terpenylcarboxyalkyl, terpenylcarbonyloxyalkyl, terpenylcarboxamido-alkyl, terpenylaminoalkyl, and terpenyloxyoalkyl.

In embodiments, one or more of rings A, B, C, and D is heterocyclic.

In embodiments, rings A, B, C, and D are non-heterocyclic.

The compounds and compositions disclosed herein are optionally prepared as salts, which advantageously makes them cationic when one or more amine groups is/are protonated. “Salt” as used herein is a broad term, and is to be given its ordinary and customary meaning to a skilled artisan (and is not to be limited to a special or customized meaning), and refers without limitation to a salt of a compound. In embodiments, the salt is an acid addition salt of the compound. Salts can be obtained by reacting a compound with inorganic acids such as hydrohalic acid (e.g., hydrochloric acid or hydrobromic acid), sulfuric acid, nitric acid, phosphoric acid, and phosphonic acid. Salts can also be obtained by reacting a compound with an organic acid such as aliphatic or aromatic carboxylic or sulfonic acids, sulfinic acids, for example formic acid, acetic acid, propionic acid, glycolic acid, pyruvic acid, malonic acid, maleic acid, fumaric acid, trifluoroacetic acid, benzoic acid, cinnamic acid, mandelic acid, succinic acid, lactic acid, malic acid, tartaric acid, citric acid, ascorbic acid, nicotinic acid, methanesulfonic acid, ethanesulfonic acid, p-toluensulfonic acid, salicylic acid, stearic acid, muconic acid, butyric acid, phenylacetic acid, phenylbutyric acid, valproic acid, 1,2-ethanedisulfonic acid, 2-hydroxyethanesulfonic acid, benzenesulfonic acid, 2-naphthalenesulfonic acid, or 1,5-naphthalenedisulfonic acid (NDSA). Salts can also be obtained by reacting a compound with a base to form a salt such as an ammonium salt, an alkali metal salt, such as a lithium, sodium or a potassium salt, an alkaline earth metal salt, such as a calcium, magnesium or aluminum salt, a salt of organic bases such as dicyclohexylamine, N-methyl-D-glucamine, tris(hydroxymethyl)methylamine, C₁-C₇ alkylamine, cyclohexyl-amine, dicyclohexylamine, triethanolamine, ethylenediamine, ethanolamine, diethanolamine, triethanolamine, tromethamine, and salts with amino acids such as arginine and lysine; or a salt of an inorganic base, such as aluminum hydroxide, calcium hydroxide, potassium hydroxide, sodium carbonate, sodium hydroxide, or the like.

In embodiments, the salt is a hydrochloride salt. In embodiments, the salt is a mono-hydrochloride salt, a di-hydrochloride salt, a tri-hydrochloride salt, or a tetra-hydrochloride salt. Additional examples of salts include sulfuric acid addition salts, sulfonic acid addition salts, disulfonic acid addition salts, 1,5-naphthalenedisulfonic acid addition salts, sulfate salts, and bisulfate salts.

“R” groups such as, without limitation, R₁, R₂, R₃, R₄, R₅, R₆. R₇, R₈, R₉, R₁₀, R₁₁, R₁₂, R₁₃, R₁₄, R₁₅. R₁₆, R₁₇, and R₁₈, represent substituents that can be attached to the sterol backbone. Unless otherwise specified, an R group may be substituted or unsubstituted.

A “ring” can be heterocyclic or carbocyclic. “Saturated” means a ring in which each atom is either hydrogenated or substituted such that the valency of each atom is filled. “Unsaturated” means a ring where the valency of each atom of the ring may not be filled with hydrogen or other substituents. For example, adjacent carbon atoms in a fused ring can be double bound to each other. Unsaturation can also include deleting at least one of the following pairs and completing the valency of the ring carbon atoms at these deleted positions with a double bond, such as R₅ and R₉; R₈ and R₁₀; and R₁₃ and R₁₄.

Where a group is “substituted” it may be substituted with one, two, three or more of the indicated substituents, which may be the same or different, each replacing a hydrogen atom. If no substituents are indicated, the indicated “substituted” group may be substituted with one or more groups individually and independently selected from alkyl, alkenyl, alkynyl, cycloalkyl, cycloalkenyl, cycloalkynyl, acylalkyl, alkoxyalkyl, aminoalkyl, amino acid, aryl, heteroaryl, heteroalicyclyl, aralkyl, heteroaralkyl, (heteroalicyclyl)alkyl, hydroxy, protected hydroxyl, alkoxy, aryloxy, acyl, mercapto, alkylthio, arylthio, cyano, halogen (e.g., F, Cl, Br, and I), thiocarbonyl, O-carbamyl, N-carbamyl, O-thiocarbamyl, N-thiocarbamyl, C-amido, N-amido, S-sulfonamido, N-sulfonamido, C-carboxy, protected C-carboxy, O-carboxy, isocyanato, thiocyanato, isothiocyanato, nitro, oxo, silyl, sulfenyl, sulfinyl, sulfonyl, haloalkyl, haloalkoxy, trihalomethanesulfonyl, trihalomethanesulfonamido, an amino, a mono-substituted amino group and a di-substituted amino group, RₐO(CH₂)ₘO—, R_{b}(CH₂)ₙO—, R_{c}(O)O(CH₂)ₚO—, and protected derivatives thereof. The substituent may be attached to the group at more than one attachment point. For example, an aryl group may be substituted with a heteroaryl group at two attachment points to form a fused multicyclic aromatic ring system. Biphenyl and naphthalene are two examples of an aryl group that is substituted with a second aryl group. A group that is not specifically labeled as substituted or unsubstituted may be considered to be either substituted or unsubstituted.

The terms “Cₐ” or “Cₐ to C_{b}” in which “a” and “b” are integers refer to the number of carbon atoms in an alkyl, alkenyl or alkynyl group, or the number of carbon atoms in the ring of a cycloalkyl, cycloalkenyl, cycloalkynyl, aryl, heteroaryl or heteroalicyclyl group. That is, the alkyl, alkenyl, alkynyl, ring of the cycloalkyl, ring of the cycloalkenyl, ring of the cycloalkynyl, ring of the aryl, ring of the heteroaryl or ring of the heteroalicyclyl can contain from “a” to “b”, inclusive, carbon atoms. Thus, for example, a “C₁ to C₄ alkyl” group refers to all alkyl groups having 1 to 4 carbons, that is, CH₃—, CH₃CH₂—, CH₃CH₂CH₂—, (CH₃)₂CH—, CH₃CH₂CH₂CH₂—, CH₃CH₂CH(CH₃)—, (CH₃)₂CHCH₂— and (CH₃)₃C—. If no “a” and “b” are designated with regard to an alkyl, alkenyl, alkynyl, cycloalkyl cycloalkenyl, cycloalkynyl, aryl, heteroaryl or heteroalicyclyl group, the broadest range described in these definitions is to be assumed.

“Alkyl” means a straight or branched hydrocarbon chain that comprises a fully saturated (no double or triple bonds) hydrocarbon group. The alkyl group may have 1 to 25 carbon atoms (whenever it appears herein, a numerical range such as “1 to 25” refers to each integer in the given range; e.g., “1 to 25 carbon atoms” means that the alkyl group may consist of 1 carbon atom, 2 carbon atoms, 3 carbon atoms, etc., up to and including 25 carbon atoms, although the present definition also covers the occurrence of the term “alkyl” where no numerical range is designated). The alkyl group may also be a medium size alkyl having 1 to 15 carbon atoms. The alkyl group could also be a lower alkyl having 1 to 6 carbon atoms. The alkyl group of the compounds may be designated as “C₄” or “C₁-C₄ alkyl” or similar designations. By way of example only, “C₁-C₄ alkyl” indicates that there are one to four carbon atoms in the alkyl chain, i.e., the alkyl chain is selected from methyl, ethyl, propyl, iso-propyl, n-butyl, iso-butyl, sec-butyl, and t-butyl. Typical alkyl groups include, but are in no way limited to, methyl, ethyl, propyl, isopropyl, butyl, isobutyl, tertiary butyl, pentyl and hexyl. The alkyl group may be substituted or unsubstituted.

“Alkenyl” means an alkyl group that contains in the straight or branched hydrocarbon chain one or more double bonds. The alkenyl group may have 2 to 25 carbon atoms (whenever it appears herein, a numerical range such as “2 to 25” refers to each integer in the given range; e.g., “2 to 25 carbon atoms” means that the alkenyl group may consist of 2, 3, or 4 carbon atoms, etc., up to and including 25 carbon atoms, although the present definition also covers the occurrence of the term “alkenyl” where no numerical range is designated). The alkenyl group may also be a medium size alkenyl having 2 to 15 carbon atoms. The alkenyl group could also be a lower alkenyl having 1 to 6 carbon atoms. The alkenyl group of the compounds may be designated as “C₄” or “C₂-C₄ alkenyl” or similar designations. An alkenyl group may be unsubstituted or substituted.

“Alkynyl” means an alkyl group that contains in the straight or branched hydrocarbon chain one or more triple bonds. The alkynyl group may have 2 to 25 carbon atoms (whenever it appears herein, a numerical range such as “2 to 25” refers to each integer in the given range; e.g., “2 to 25 carbon atoms” means that the alkynyl group may consist of 2, 3, or 4 carbon atoms, etc., up to and including 25 carbon atoms, although the present definition also covers the occurrence of the term “alkynyl” where no numerical range is designated). The alkynyl group may also be a medium size alkynyl having 2 to 15 carbon atoms. The alkynyl group could also be a lower alkynyl having 2 to 6 carbon atoms. The alkynyl group of the compounds may be designated as “C₄” or “C₂-C₄ alkynyl” or similar designations. An alkynyl group may be unsubstituted or substituted.

“Aryl” means a carbocyclic (all carbon) monocyclic or multicyclic aromatic ring system (including fused ring systems where two carbocyclic rings share a chemical bond) that has a fully delocalized pi-electron system throughout all the rings. The number of carbon atoms in an aryl group can vary. For example, the aryl group can be a C₆-C₁₄ aryl group, a C₆-C₁₀ aryl group, or a C₆ aryl group (although the definition of C₆-C₁₀ aryl covers the occurrence of “aryl” when no numerical range is designated). Examples of aryl groups include, but are not limited to, benzene, naphthalene and azulene. An aryl group may be substituted or unsubstituted.

“Aralkyl” and “aryl(alkyl)” mean an aryl group connected, as a substituent, via a lower alkylene group. The aralkyl group may have 6 to 20 carbon atoms (whenever it appears herein, a numerical range such as “6 to 20” refers to each integer in the given range; e.g., “6 to 20 carbon atoms” means that the aralkyl group may consist of 6 carbon atom, 7 carbon atoms, 8 carbon atoms, etc., up to and including 20 carbon atoms, although the present definition also covers the occurrence of the term “aralkyl” where no numerical range is designated). The lower alkylene and aryl group of an aralkyl may be substituted or unsubstituted. Examples include but are not limited to benzyl, 2-phenylalkyl, 3-phenylalkyl, and naphthylalkyl.

“Lower alkylene groups” mean a C₁-C₂₅ straight-chained alkyl tethering groups, such as —CH₂— tethering groups, forming bonds to connect molecular fragments via their terminal carbon atoms. Examples include but are not limited to methylene (—CH₂—), ethylene (—CH₂CH₂—), propylene (—CH₂CH₂CH₂—), and butylene (—CH₂CH₂CH₂CH₂—). A lower alkylene group can be substituted by replacing one or more hydrogen of the lower alkylene group with a substituent(s) listed under the definition of “substituted.”

“Cycloalkyl” means a completely saturated (no double or triple bonds) mono- or multi-cyclic hydrocarbon ring system. When composed of two or more rings, the rings may be joined together in a fused fashion. Cycloalkyl groups can contain 3 to 10 atoms in the ring(s) or 3 to 8 atoms in the ring(s). A cycloalkyl group may be unsubstituted or substituted. Typical cycloalkyl groups include, but are in no way limited to, cyclopropyl, cyclobutyl, cyclopentyl, cyclohexyl, cycloheptyl and cyclooctyl.

“Cycloalkenyl” means a mono- or multi-cyclic hydrocarbon ring system that contains one or more double bonds in at least one ring; although, if there is more than one, the double bonds cannot form a fully delocalized pi-electron system throughout all the rings (otherwise the group would be “aryl,” as defined herein). When composed of two or more rings, the rings may be connected together in a fused fashion. A cycloalkenyl group may be unsubstituted or substituted.

“Cycloalkynyl” means a mono- or multi-cyclic hydrocarbon ring system that contains one or more triple bonds in at least one ring. If there is more than one triple bond, the triple bonds cannot form a fully delocalized pi-electron system throughout all the rings. When composed of two or more rings, the rings may be joined together in a fused fashion. A cycloalkynyl group may be unsubstituted or substituted.

“Alkoxy” or “alkyloxy” mean the formula —OR wherein R is an alkyl, an alkenyl, an alkynyl, a cycloalkyl, a cycloalkenyl or a cycloalkynyl as defined above. Examples of alkoxys are methoxy, ethoxy, n-propoxy, 1-methylethoxy (isopropoxy), n-butoxy, iso-butoxy, sec-butoxy and tert-butoxy. An alkoxy may be substituted or unsubstituted.

“Acyl” means a hydrogen, alkyl, alkenyl, alkynyl, aryl, or heteroaryl connected, as substituents, via a carbonyl group, such as —(C═O)—R. Examples include formyl, acetyl, propanoyl, benzoyl, and acryl. An acyl may be substituted or unsubstituted.

“Alkoxyalkyl” or “alkyloxyalkyl” mean an alkoxy group connected, as a substituent, via a lower alkylene group. Examples include alkyl-O-alkyl- and alkoxy-alkyl- with the terms alkyl and alkoxy defined herein.

“Hydroxyalkyl” means an alkyl group in which one or more of the hydrogen atoms are replaced by a hydroxy group. Exemplary hydroxyalkyl groups include but are not limited to, 2-hydroxyethyl, 3-hydroxypropyl, 2-hydroxypropyl, and 2,2-dihydroxyethyl. A hydroxyalkyl may be substituted or unsubstituted.

“Haloalkyl” means an alkyl group in which one or more of the hydrogen atoms are replaced by a halogen (e.g., mono-haloalkyl, di-haloalkyl and tri-haloalkyl). Examples include chloromethyl, fluoromethyl, difluoromethyl, trifluoromethyl and 1-chloro-2-fluoromethyl, 2-fluoroisobutyl. A haloalkyl may be substituted or unsubstituted.

“Amino” means “—NH₂”.

“Hydroxy” means “—OH”.

“Cyano” means “—CN”.

“Carbonyl” or “oxo” mean “—C═O”.

“Azido” means “—N₃”.

“Aminoalkyl” means an amino group connected, as a substituent, via a lower alkylene group. Examples include H₂N-alkyl- with the term alkyl defined herein.

“Alkylcarboxyalkyl” means an alkyl group connected, as a substituent, to a carboxy group that is connected, as a substituent, to an alkyl group. Examples include alkyl-(C═O)—O-alkyl- and alkyl-O—(C═O)-alkyl- with the term alkyl as defined herein.

“Alkylaminoalkyl” means an alkyl group connected, as a substituent, to an amino group that is connected, as a substituent, to an alkyl group. Examples include alkyl-NH-alkyl- with the term alkyl as defined herein.

“Dialkylaminoalkyl” and “di(alkyl)aminoalkyl” mean two alkyl groups connected, each as a substituent, to an amino group that is connected, as a substituent, to an alkyl group. Examples include

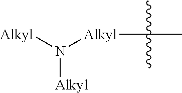

with the term alkyl as defined herein.

“Alkylaminoalkylamino” means an alkyl group connected, as a substituent, to an amino group that is connected, as a substituent, to an alkyl group that is connected, as a substituent, to an amino group. Examples include alkyl-NH-alkyl-NH— with the term alkyl as defined herein.

“Alkylaminoalkylaminoalkylamino” means an alkyl group connected, as a substituent, to an amino group that is connected, as a substituent, to an alkyl group that is connected, as a substituent, to an amino group that is connected, as a substituent, to an alkyl group. Examples include alkyl-NH-alkyl-NH-alkyl- with the term alkyl as defined herein.

“Arylaminoalkyl” means an aryl group connected, as a substituent, to an amino group that is connected, as a substituent, to an alkyl group. Examples include aryl-NH-alkyl- with the terms aryl and alkyl as defined herein.

“Aminoalkyloxy” means an amino group connected, as a substituent, to an alkyloxy group. Examples include H₂N-alkyl-O— and H₂N-alkoxy- with the terms alkyl and alkoxy as defined herein.

“Aminoalkyloxyalkyl” means an amino group connected, as a substituent, to an alkyloxy group connected, as a substituent, to an alkyl group. Examples include H₂N-alkyl-O-alkyl- and H₂N-alkoxy-alkyl- with the terms alkyl and alkoxy as defined herein.

“Aminoalkylcarboxy” means an amino group connected, as a substituent, to an alkyl group connected, as a substituent, to a carboxy group. Examples include H₂N-alkyl-(C═O)—O— and H₂N-alkyl-O—(C═O)— with the term alkyl as defined herein.

“Aminoalkylaminocarbonyl” means an amino group connected, as a substituent, to an alkyl group connected, as a substituent, to an amino group connected, as a substituent, to a carbonyl group. Examples include H₂N-alkyl-NH—(C═O)— with the term alkyl as defined herein.

“Aminoalkylcarboxamido” means an amino group connected, as a substituent, to an alkyl group connected, as a substituent, to a carbonyl group connected, as a substituent to an amino group. Examples include H₂N-alkyl-(C═O)—NH— and H₂N-alkyl-NH—(C═O)— with the term alkyl as defined herein.

“Azidoalkyloxy” means an azido group connected as a substituent, to an alkyloxy group. Examples include N₃-alkyl-O— and N₃-alkoxy- with the terms alkyl and alkoxy as defined herein.

“Cyanoalkyloxy” means a cyano group connected as a substituent, to an alkyloxy group. Examples include NC-alkyl-O— and NC-alkoxy- with the terms alkyl and alkoxy as defined herein.

“Sulfenyl” means “—SR” in which R can be hydrogen, alkyl, alkenyl, alkynyl, cycloalkyl, cycloalkenyl, cycloalkynyl, aryl, heteroaryl, heteroalicyclyl, aralkyl, or (heteroalicyclyl)alkyl. A sulfenyl may be substituted or unsubstituted.

“Sulfinyl” means “—(S═O)—R” in which R can be the same as defined with respect to sulfenyl. A sulfinyl may be substituted or unsubstituted.

“Sulfonyl” means “—(S═O)—OR” in which R can be the same as defined with respect to sulfenyl. A sulfonyl may be substituted or unsubstituted.

“O-carboxy” means “R—(C═O)—O—” in which R can be hydrogen, alkyl, alkenyl, alkynyl, cycloalkyl, cycloalkenyl, cycloalkynyl, aryl, heteroaryl, heteroalicyclyl, aralkyl, or (heteroalicyclyl)alkyl, as defined herein. An O-carboxy may be substituted or unsubstituted.

“Ester” and “C-carboxy” mean “—(C═O)—OR” in which R can be the same as defined with respect to O-carboxy. An ester and C-carboxy may be substituted or unsubstituted.

“Thiocarbonyl” means “—(C═S)—R” in which R can be the same as defined with respect to O-carboxy. A thiocarbonyl may be substituted or unsubstituted.

“Trihalomethanesulfonyl” means “X₃CSO₂—” wherein X is a halogen.

“S-sulfonamido” means “—SO₂N(RARB)” in which RA and RB can be independently hydrogen, alkyl, alkenyl, alkynyl, cycloalkyl, cycloalkenyl, cycloalkynyl, aryl, heteroaryl, heteroalicyclyl, aralkyl, or (heteroalicyclyl)alkyl. An S-sulfonamido may be substituted or unsubstituted.

“N-sulfonamido” means “RSO₂N(RA)-” in which R and RA can be independently hydrogen, alkyl, alkenyl, alkynyl, cycloalkyl, cycloalkenyl, cycloalkynyl, aryl, heteroaryl, heteroalicyclyl, aralkyl, or (heteroalicyclyl)alkyl. An N-sulfonamido may be substituted or unsubstituted.

“O-carbamyl” and “urethanyl” mean “—O—(C═O)—N(RARB)” in which RA and RB can be independently hydrogen, alkyl, alkenyl, alkynyl, cycloalkyl, cycloalkenyl, cycloalkynyl, aryl, heteroaryl, heteroalicyclyl, aralkyl, or (heteroalicyclyl)alkyl. An O-carbamyl or urethanyl may be substituted or unsubstituted.

“N-carbamyl” means “RO—(C═O)—N(RA)-” in which R and RA can be independently hydrogen, alkyl, alkenyl, alkynyl, cycloalkyl, cycloalkenyl, cycloalkynyl, aryl, heteroaryl, heteroalicyclyl, aralkyl, or (heteroalicyclyl)alkyl. An N-carbamyl may be substituted or unsubstituted.

“O-thiocarbamyl” means “—O—(C═S)—N(RARB)” in which RA and RB can be independently hydrogen, alkyl, alkenyl, alkynyl, cycloalkyl, cycloalkenyl, cycloalkynyl, aryl, heteroaryl, heteroalicyclyl, aralkyl, or (heteroalicyclyl)alkyl. An O-thiocarbamyl may be substituted or unsubstituted.

“N-thiocarbamyl” means “RO—(C═S)—N(RA)-” in which R and RA can be independently hydrogen, alkyl, alkenyl, alkynyl, cycloalkyl, cycloalkenyl, cycloalkynyl, aryl, heteroaryl, heteroalicyclyl, aralkyl, or (heteroalicyclyl)alkyl. An N-thiocarbamyl may be substituted or unsubstituted.

“C-amido” means “—(C═O)—N(RARB)” in which RA and RB are independently hydrogen, alkyl, alkenyl, alkynyl, cycloalkyl, cycloalkenyl, cycloalkynyl, aryl, heteroaryl, heteroalicyclyl, aralkyl, or (heteroalicyclyl)alkyl. A C-amido may be substituted or unsubstituted.

“N-amido” means “R—(C═O)—N(RA)-” in which R and RA are independently hydrogen, alkyl, alkenyl, alkynyl, cycloalkyl, cycloalkenyl, cycloalkynyl, aryl, heteroaryl, heteroalicyclyl, aralkyl, or (heteroalicyclyl)alkyl. An N-amido may be substituted or unsubstituted.

“Guanidinoalkyloxy” means a guanidinyl group connected, as a substituent, to an alkyloxy group. Examples are

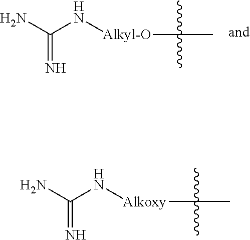

with the terms alkyl and alkoxy as defined herein.

“Guanidinoalkylcarboxy” means a guanidinyl group connected, as a substituent, to an alkyl group connected, as a substituent, to a carboxy group. Examples are

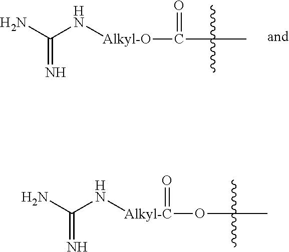

with the term alkyl as defined herein.

“Quaternary ammonium alkylcarboxy” means a quaternized amino group connected, as a substituent, to an alkyl group connected, as a substituent, to a carboxy group. Examples are

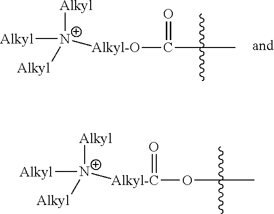

with the term alkyl as defined herein.

“Halogen atom” and “halogen” mean any one of the radio-stable atoms of column 7 of the Periodic Table of the Elements, such as, fluorine, chlorine, bromine and iodine.

Where the number of substituents is not specified (e.g. haloalkyl), there may be one or more substituents present. For example, “haloalkyl” may include one or more of the same or different halogens.

“Amino acid” means any amino acid (both standard and non-standard amino acids), including, but not limited to, α-amino acids, β-amino acids, γ-amino acids and δ-amino acids. Examples of suitable amino acids include, but are not limited to, alanine, asparagine, aspartate, cysteine, glutamate, glutamine, glycine, proline, serine, tyrosine, arginine, histidine, isoleucine, leucine, lysine, methionine, phenylalanine, threonine, tryptophan and valine. Additional examples of suitable amino acids include, but are not limited to, ornithine, hypusine, 2-aminoisobutyric acid, dehydroalanine, γ-aminobutyric acid, citrulline, β-alanine, α-ethyl-glycine, α-propyl-glycine and norleucine.

A “linking group” is a divalent moiety used to link one steroid to another steroid. In embodiments, the linking group is used to link a first CSA with a second CSA (which may be the same or different). An example of a linking group is (C₁-C₁₀) alkyloxy-(C₁-C₁₀) alkyl.

“P.G.” or “protecting group” or “protecting groups” mean any atom or group of atoms that is added to a molecule in order to prevent existing groups in the molecule from undergoing unwanted chemical reactions. Examples of protecting group moieties are described in T. W. Greene and P. G. M. Wuts, Protective Groups in Organic Synthesis, 3. Ed. John Wiley & Sons, 1999, and in J. F. W. McOmie, Protective Groups in Organic Chemistry Plenum Press, 1973, both of which are hereby incorporated by reference for the limited purpose of disclosing suitable protecting groups. The protecting group moiety may be chosen in such a way, that they are stable to certain reaction conditions and readily removed at a convenient stage using methodology known from the art. A non-limiting list of protecting groups include benzyl; substituted benzyl; alkylcarbonyls and alkoxycarbonyls (e.g., t-butoxycarbonyl (BOC), acetyl, or isobutyryl); arylalkylcarbonyls and arylalkoxycarbonyls (e.g., benzyloxycarbonyl); substituted methyl ether (e.g. methoxymethyl ether); substituted ethyl ether; substituted benzyl ether; tetrahydropyranyl ether; silyls (e.g., trimethylsilyl, triethylsilyl, triisopropylsilyl, t-butyldimethylsilyl, tri-iso-propylsilyloxymethyl, [2-(trimethylsilyl) ethoxy]methyl or t-butyldiphenylsilyl); esters (e.g. benzoate ester); carbonates (e.g. methoxymethylcarbonate); sulfonates (e.g. tosylate or mesylate); acyclic ketal (e.g. dimethyl acetal); cyclic ketals (e.g., 1,3-dioxane, 1,3-dioxolanes, and those described herein); acyclic acetal; cyclic acetal (e.g., those described herein); acyclic hemiacetal; cyclic hemiacetal; cyclic dithioketals (e.g., 1,3-dithiane or 1,3-dithiolane); orthoesters (e.g., those described herein) and triarylmethyl groups (e.g., trityl; monomethoxytrityl (MMTr); 4,4′-dimethoxytrityl (DMTr); 4,4′,4″-trimethoxytrityl (TMTr); and those described herein). Amino-protecting groups are known to those skilled in the art. In general, the species of protecting group is not critical, provided that it is stable to the conditions of any subsequent reaction(s) on other positions of the compound and can be removed at the appropriate point without adversely affecting the remainder of the molecule. In addition, a protecting group may be substituted for another after substantive synthetic transformations are complete. Clearly, where a compound differs from a compound disclosed herein only in that one or more protecting groups of the disclosed compound has been substituted with a different protecting group, that compound is within the disclosure.

1. An adhesive medical film incorporating one or more cationic steroidal antimicrobial (CSA) compounds, comprising:
a polymer adhesive in which one or more CSA compounds are distributed; and
optionally, a backing substrate upon which the polymer adhesive is disposed, the backing substrate being free of CSA compounds,
wherein the adhesive medical film is formed as a tape, sheet, or dressing configured for contact with dermal tissue, and
wherein the adhesive medical film, when used, reduces microbial colonization and/or growth as compared to the same adhesive medical film without incorporated CSA compounds.

2. The adhesive medical film of claim 1, wherein the one or more CSA compounds comprise a CSA compound that includes cationic groups linked to a sterol backbone with non-hydrolysable linkages.

3. The adhesive medical film of claim 2, wherein the lap shear strength of the polymer adhesive is increased as compared to the same polymer adhesive without incorporated CSA compounds, such as wherein the lap shear strength of the polymer adhesive is increased by at least about 1.5 times and up to about 4.5 times as compared to the same polymer adhesive without incorporated CSA compounds.

4. The adhesive medical film of claim 2, wherein the non-hydrolysable linkages are ether linkages.

5. The adhesive medical film of claim 4, wherein the one or more CSA compounds comprise CSA-131.

6. The adhesive medical film of claim 1, wherein the one or more CSA compounds comprise a CSA compound that includes cationic groups linked to a sterol backbone with hydrolysable linkages.

7. The adhesive medical film of claim 6, wherein the hydrolysable linkages are ester linkages.

8. The adhesive medical film of claim 7, wherein the one or more CSA compounds comprise CSA-44.

9. The adhesive medical film of claim 1, wherein the polymer adhesive comprises a polyacrylate polymer formed from a set of one or more (meth)acrylate monomers.

10. The adhesive medical film of claim 9, wherein the one or more (meth)acrylate monomers comprise an alkyl (meth)acrylate, a cycloalkyl (meth)acrylate, a di(meth)acrylate, a (meth)acrylic acid, or combination thereof.

11. The adhesive medical film of claim 1, wherein the polymer adhesive is formulated as a pressure-sensitive adhesive (PSA).

12. The adhesive medical film of claim 1, wherein the backing substrate comprises a woven fabric, non-woven fabric, polymer film, polymer foam, or combination thereof.

13. The adhesive medical film of claim 1, wherein the polymer adhesive comprises a polysiloxane.

14. The adhesive medical film of claim 13, wherein the polymer adhesive comprises a first side and a second side, wherein the first side comprises capped polysiloxane and the second side comprises uncapped polysiloxane.

15. The adhesive medical film of claim 1, wherein the polymer adhesive has an average thickness of about 50 μm to about 500 μm, or such as about 75 μm to about 300 μm, or such as about 100 μm to about 200 μm, or such as about 120 μm to about 150 μm, or a thickness within a range with endpoints selected from any two of the foregoing values.

16. The adhesive medical film of claim 1, wherein the polymer adhesive includes the one or more CSA compounds at a concentration of about 4.8 μg/cm³ to about 36 μg/cm³, or about 6 μg/cm³ to about 24 μg/cm³, or about 9 μg/cm³ to about 18 μg/cm³, or about 16.8 μg/cm³, or within a range with endpoints selected from any two of the foregoing values.

17. A method of manufacturing an adhesive medical film incorporating one or more cationic steroidal antimicrobial (CSA) compounds, comprising:
mixing one or more CSA compounds with an organic solvent to form a CSA liquid;
mixing the CSA liquid with a polymer adhesive composition, wherein the polymer adhesive composition comprises polyacrylate, to form a CSA adhesive mixture;
spreading the CSA adhesive mixture onto a releasable substrate;
allowing the CSA adhesive mixture to dry to form a polymer adhesive incorporating the one or more CSA compounds; and
optionally, adding a backing substrate to an upper surface of the polymer adhesive after allowing the CSA adhesive mixture to dry.

18. The method of claim 17, wherein the one or more CSA compounds are included in an amount such that the CSA liquid includes the one or more CSA compounds at a concentration of about 20% (w/v) to about 50% (w/v), or about 25% (w/v) to about 45% (w/v), or about 30% (w/v) to about 40% (w/v), or about 35% (w/v), or within a range with endpoints selected from any two of the foregoing values.

19. The method of claim 17, wherein the organic solvent comprises an organic alcohol, such as ethanol.

20. The method of claim 17, wherein the CSA adhesive mixture comprises a weight ratio of polyacrylate solids to the one or more CSA compounds of about 1.5 to about 3.5, such as about 2 to about 3, or such as about 2.5, or a ratio within a range with endpoints selected from any two of the foregoing values.

21. The method of claim 17, wherein the CSA adhesive mixture is spread to a thickness of about 100 μm to about 350 μm, such as about 150 μm to about 250 μm, or such as about 200 μm, or a thickness within a range with endpoints selected from any two of the foregoing values.

22. The method of claim 17, used to form an adhesive medical film as in any one of claims 9 to 16.

23. A method of manufacturing an adhesive medical film incorporating one or more cationic steroidal antimicrobial (CSA) compounds, comprising:
mixing one or more CSA compounds with an organic alcohol to form a CSA liquid;
soaking a silicone film material in the CSA liquid such that the silicone film material swells and takes up at least a portion of the one or more CSA compounds of the CSA liquid; and
optionally, sonicating the CSA liquid while the silicone film material is in the CSA liquid.

24. The method of claim 23, wherein the one or more CSA compounds are included in an amount such that the CSA liquid includes the one or more CSA compounds at a concentration of about 0.5% (w/v) to about 5% (w/v), such as about 0.75% (w/v) to about 2.5% (w/v), or such as about 1% (w/v), or within a range with endpoints selected from any two of the foregoing values.

25. The method of claim 23, wherein the silicone film material comprises a first side and a second side, wherein the first side comprises capped polysiloxane and the second side comprises uncapped polysiloxane.

26. The method of claim 23, used to form an adhesive medical film as in any one of claims 13 to 16.

27. A method of using an adhesive medical film as in claim 1, comprising:
applying the adhesive medical film such that at least a portion contacts dermal tissue of a subject; and
the adhesive medical film reducing microbial colonization and/or growth as compared to the same adhesive medical film without incorporated CSA compounds.

28. The method of claim 27, wherein the adhesive medical film provides at least a 3-log reduction in colonization of gram-positive bacteria, gram-negative bacteria, and pathogenic fungi following 24 hours of surface exposure thereto, as compared to the same adhesive medical film without incorporated CSA compounds.

29. The method of claim 27, wherein the adhesive medical film reduces microbial growth of gram-positive bacteria, gram-negative bacteria, and pathogenic fungi, when exposed thereto, for at least 6 days, as compared to the same adhesive medical film without incorporated CSA compounds.

30. The method of claim 29, wherein the adhesive medical film reduces growth of MRSA, when exposed thereto, for at least about 3 weeks, such as up to about 68 days, as compared to the same adhesive medical film without the incorporated CSA compounds.
